(19) United States
(12) Patent Application Publication
(10) Pub. No.: US 2026/0308103 A1
(43) Pub. Date: Oct. 8, 2026

(54) COBALT-CATALYZED CARBONYLATIVE ALKENE HYDROFUNCTIONALIZATIONS PROMOTED BY VISIBLE LIGHT

(71) Applicant: THE UNIVERSITY OF NORTH CAROLINA AT CHAPEL HILL, Chapel Hill, NC (US)

(72) Inventors: Erik Alexanian, Chapel Hill, NC (US); Mason Faculak, Chapel Hill, NC (US); Alexander Veatch, Greensboro, NC (US)

(21) Appl. No.: 19/483,351

(22) PCT Filed: May 22, 2024
(86) PCT No.: PCT/US2024/030480

§ 371 Date: Nov. 11, 2025

Related U.S. Application Data
(60) Provisional application No. 63/468,573, filed on May 24, 2023.

(51) Int. Cl. C07C 209/28; C07D 209/16; C07D 211/46; C07D 295/023; C07D 295/205

(52) U.S. Cl. CPC C07C 209/28; C07D 209/16; C07D 211/46; C07D 295/023; C07D 295/205

(57) ABSTRACT

The present disclosure relates to the general synthesis of amides, amines, esters, ethers, carboxylic acids, aldehydes and alcohols from alkene feedstock under mild conditions using a cobalt catalyst in a carbon monoxide atmosphere and light.

### GOVERNMENT SUPPORT

This invention was made with government support under Grant No. GM131708 awarded by the National Institutes of Health. The government has certain rights in the invention.

### TECHNICAL FIELD

The present disclosure relates to the general synthesis of amides, amines, esters, carboxylic acids, ethers, alcohols and aldehydes from alkene feedstocks under mild conditions using a cobalt catalyst under a carbon monoxide atmosphere with light.

### BACKGROUND

Carbonylative hydrofunctionalizations of alkenes represent a 100% atom economical method towards synthesizing carbonyl-containing small molecules. With the abundance of carbonyl functionalities like amides, esters, aldehydes and carboxylic acids in industrially relevant chemicals and the commercial availability of alkene starting materials, these carbonylative transformations provide a method of obtaining valuable chemicals efficiently from synthetic feedstocks (Scheme 1).

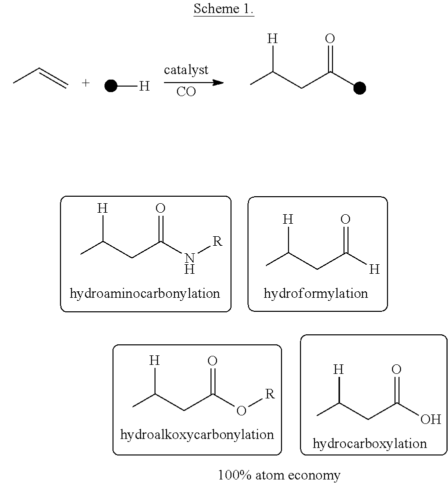

### Various Carbonylative Hydrofunctionalizations

Hydroaminocarbonylation—the coupling of alkenes, amines and carbon monoxide—remains as a challenge in catalysis due to the strong Lewis basicity of alkylamines poisoning metal catalysts and inhibiting requisite catalytic intermediates (i.e., metal hydrides). Hydroformylation—the coupling of alkenes, hydrogen and carbon monoxide—is a classic example of a carbonylative hydrofunctionalization which has been studied and used on large scale industrial synthesis to make aldehydes for almost a century. However, these systems typically employ harsh reaction temperatures and pressures, and only systems using rare-Earth transition metal catalysts with carefully designed ligands are able to control regioselectivity. Hyrdoalkoxycarbonylation—the coupling of alkenes, alcohols and carbon monoxide—suffers from the same challenges as hydroformylation and requires solvent quantities of alcohol nucleophile which limits the reaction scope to simple alcohols.

Sequential reductive catalysis after alkene carbonylative hydrofunctionalizations allow for the generation of other valuable small molecules. For example, reductive hydroformylation and hydroaminomethylation use hydrogen gas in a sequential reduction step after hydroformylation to produce alcohols and amines, respectively (Scheme 2). Current methods using Earth-abundant metal catalysts require high temperatures and pressures of synthesis gas which limits their substrate scopes.

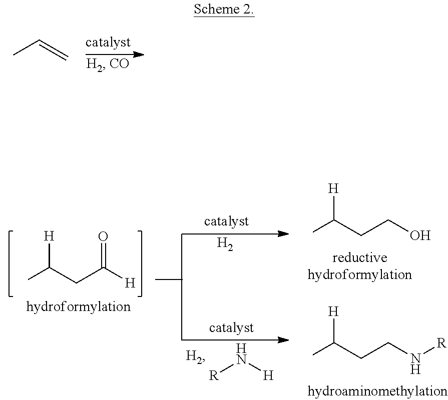

### Current Methods of Sequential Reduction Catalysis for Carbonylative Hydrofunctionalization

Thus, there is a great need for the development of carbonylative hydrofunctionalization processes that can be widely applied to alkenes using Earth-abundant metal catalysts under mild conditions to efficiently synthesis carbonyl-containing compounds or their reduced variants.

### SUMMARY

The methods disclosed herein can be used to prepare amides and amines from an exceptionally broad scope across both alkene and amine components with high reaction chemo- and regioselectivity.

One aspect of the current disclosure relates to a method of making an amide, the methods comprising contacting an olefin with an amine in the presence of a cobalt catalyst under a carbon monoxide atmosphere while being exposed to light. In some embodiments, the amine employed in this methods is a compound of Formula I:

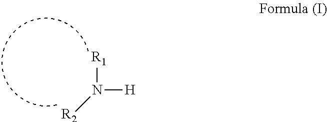

wherein R₁ and R₂ are independently selected from the group consisting of hydrogen, alkyl, cycloalkyl, heterocycloalkyl, aryl, heteroaryl, alkenyl, alkynyl, haloalkyl, heteroalkyl, bicyclic cycloalkyl, bicyclic heterocycloalkyl, cycloalkenyl, —(CH₂)ₘaryl, and —(CH₂)ₚheteroaryl, wherein m and p are integers independently selected from about 1 to about 20 atoms; and wherein [symbol] represents a single bond, if present, between R₁ and R₂. In some embodiments, R₁ and/or R₂ can be substituted with one or more substituents.

Another aspect of the current disclosure is directed to a method of making an amine by contacting the amide obtained in the above-described methods with a reducing agent to obtain the corresponding amine.

Another aspect of the current disclosure is directed towards using the above-described methods for the preparation of esters, alcohols, or ethers from couplings with alcohols.

Another aspect of the current disclosure is directed towards using the above-described methods for the preparation of carboxylic acids from couplings with water.

Another aspect of the current disclosure is directed towards using the above-described methods for the preparation of aldehydes from couplings of alkenes with hydrogen.

Another aspect of the current disclosure is directed towards using the above-described methods for the transformation of alkynes to form acrylamides and acrylates from coupling with amines and alcohols, respectively.

### DESCRIPTION OF THE DRAWINGS

FIG. 1 is a scheme showing a proposed catalytic cycle for the hydroaminocarbonylation;

FIG. 2 is a scheme showing representative cobalt-catalyzed hydroaminocarbonylations, wherein the yields are of isolated amide products. Percent selectivity provided in examples involving minor regioisomers. ^{a}2 equiv alkene. ^{b}5 equiv amine and 5 mol % Co₂(CO)₈. ^{c}4.5 equiv NH₃, iPrOH solvent.

FIG. 3A is a scheme showing various products prepared using a variety of alkenes in cobalt-catalyzed hydroaminocarbonylations, wherein the yields are of isolated amide products. ^{a}2 equiv alkene. ^{b}5 equiv amine and 5 mol % Co₂(CO)₈. ^{c}4.5 equiv NH₃, iPrOH solvent.

FIG. 3B is a scheme showing various products prepared using a variety of amines in cobalt-catalyzed hydroaminocarbonylations, wherein the yields are of isolated amide products. ^{a}4.5 equiv NH₃, iPrOH solvent.

FIG. 4 is a scheme showing various amine products prepared using a variety of amines and alkenes in catalyzed hydroaminomethylations, wherein the yields are of isolated amine products. ^{a}2 equiv alkene.

FIG. 5 is a scheme showing various products prepared using a variety of alkenes in cobalt-catalyzed hydroalkoxycarbonylations. Yields determined by analysis of crude ¹H NMR using hexamethyldisiloxane as an internal standard. ^{a}4 mol % additive A1, and 5 atm CO. ^{b}Room temperature.

FIG. 6 is a scheme showing various products prepared using a variety of alcohols in cobalt-catalyzed hydroalkoxycarbonylations. Yields determined by analysis of crude ¹H NMR using hexamethyldisiloxane as an internal standard.

FIG. 7 is a scheme showing various cobalt-catalyzed hydroaminocarbonylations to prepare cyclic products, wherein the yields are of isolated amide products. Endo:exo ratio determined by ¹H NMR analysis of crude reaction mixture and reported in parentheses. ^{a}HCl salt of amine and piperidine (1.75 equiv) were used. ^{b}48 hours. ^{c}72 hours.

FIG. 8 is a scheme showing various cobalt-catalyzed hydroalkoxycarbonylations to prepare cyclic products, wherein the yields are of isolated, major ester products. Endo:exo ratio determined by ¹H NMR analysis of crude reaction mixture and reported in parentheses. ^{a}90° C. ^{b}Endo:exo ratio determined via isolation. ^{c}Starting material was a 1.2:1 mixture of syn:anti diastereomers.

FIG. 9 is a scheme showing various cobalt-catalyzed hydroformylations promoted by visible light. Ratio of aldehydes:(hemi)acetals reported in parentheses. ^{a}Yields determined by analysis of crude ¹H NMR using hexamethyldisiloxane as an internal standard. ^{b}5 mol % Co₂(CO)₈. ^{c}The only observed product was 1,1-diethoxy-3,4-dimethylpentane.

FIG. 10 is a picture showing a pressure tube and Kessil light irradiation on sliced aluminum heating mantle to provide additional heat for hydroaminocarbonylation (covered with cardboard box to prevent stray irradiation). The temperature of the reaction mixture was evaluated with an open tube containing water and measured at 90° C.

FIG. 11 is a picture showing a pressure tube and Kessil light setup encased in an orange acrylic box to prevent stray irradiation. The temperature of the reaction mixture was unchanged (ca. 25° C.), despite an increase of temperature within the enclosure.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Disclosed herein are catalytic methods that furnish amides and amines from fundamental synthetic feedstocks utilizing carbonylative coupling chemistry. Carbonylative transformations coupling alkenes and amines in the presence of earth-abundant metals and proceeding at low temperatures and pressures are disclosed. Specifically, hydroaminocarbonylation of alkenes using an inexpensive cobalt catalyst under carbon monoxide atmosphere and exposed to light render the wide scope of amide-containing compounds, which can be reduced in situ to render the corresponding amine-containing compounds. It was surprising and unexpected that such a transformation can be observed under these reaction conditions. In addition, it was surprising and unexpected to observe that the methods disclosed herein can also be applied to furnish aldehydes, ester, carboxylic acid, alcohol, and ether-containing compounds from synthetic feedstocks utilizing essentially the same carbonylative coupling chemistry. Lastly, it was surprising and unexpected to observe that the disclosed methods can be expanded to using alkynes instead of olefins to render acrylamides and acrylates as will be discussed in more detail below.

### A. Definitions

Listed below are definitions of various terms used to describe this invention. These definitions apply to the terms as they are used throughout this specification, unless otherwise limited in specific instances, either individually or as part of a larger group.

As used in the specification and the appended claims, the singular forms “a,” “an” and “the” include plural referents unless the context clearly dictates otherwise. Thus, for example, reference to “an alkyl group” or “a polymer” includes mixtures of two or more such alkyl groups or polymers.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

References in the specification and concluding claims to parts by weight of a particular element or component in a composition denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compositions.

A weight percent (wt %) of a component, unless specifically stated to the contrary, is based on the total weight of the composition in which the component is included.

Throughout this specification and the claims, the words “comprise,” “comprises,” and “comprising” are used in a nonexclusive sense, except where the context requires otherwise. It is understood that embodiments described herein include “consisting of” and/or “consisting essentially of” embodiments.

As used herein, the terms “increase,” “increases,” “increased,” “increasing”, “improve”, “enhance”, and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more.

As used herein, the “contacting” refers to reagents in close proximity so that a reaction may occur.

As used herein, “ambient temperature” or “room temperature” refers to a temperature in the range of about 20° C. to about 25° C.

As used herein, the term “alkyl” refers to a straight or branched chain hydrocarbon containing from 1 to 20 carbon atoms. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. These groups may be substituted with groups selected from halo (e.g., haloalkyl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocycloalkyloxy, mercapto, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, ester, amide, nitro, or cyano.

The term “cycloalkyl” refers to a hydrocarbon 3-10 membered monocyclic or 7-14 membered bicyclic ring system having at least one saturated ring or having at least one non-aromatic ring, wherein the non-aromatic ring may have some degree of unsaturation. Cycloalkyl groups can range from (C₃-C₁₀)cycloalkyl, (C₃-C₈)cycloalkyl, (C₃-C₆)cycloalkyl. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

As used herein, the term “alkenyl” refers to unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl or decenyl), branched-chain alkenyl groups and cycloalkenyl (alicyclic) groups (cyclopropenyl, cyclopentenyl, cyclohexenyl, cycloheptenyl or cyclooctenyl) groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group with 10 or fewer carbon atoms in its backbone (e.g., C₂-C₁₀ for straight chain, C₃-C₁₀ for branched chain) is used. Likewise, cycloalkenyl groups may have from 3-8 carbon atoms in their ring structure, and more preferably have 5 or 6 carbons in the ring structure. The term C₂-C₁₀ includes alkenyl groups containing 2 to 10 carbon atoms.

As used herein, the term “heteroaryl” or “heteroaromatic” refers to a monovalent aromatic radical of 5- or 6-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl (including, for example, 3-amino-1,2-4-triazole or 3-mercapto-1,2,4-triazole), pyrazinyl (including, for example, aminopyrazine), tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The heteroaryl groups are thus, in some embodiments, monocyclic or bicyclic. Heteroaryl groups are optionally substituted independently with one or more substituents described herein.

As used herein, the term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

As used herein, the term “substituted” refers to a moiety (such as an alkyl group), wherein the moiety is bonded to one or more additional organic radicals. In some embodiments, the substituted moiety comprises 1, 2, 3, 4, or 5 additional substituent groups or radicals. Suitable organic substituent radicals include, but are not limited to, hydroxyl, amino, mono-substituted amino, di-substituted amino, mercapto, alkylthiol, alkoxy, substituted alkoxy or haloalkoxy radicals, wherein the terms are defined herein. Unless otherwise indicated herein, the organic substituents can comprise from 1 to 4 or from 5 to 8 carbon atoms. When a substituted moiety is bonded thereon with more than one substituent radical, then the substituent radicals may be the same or different.

As used herein, the term “alkoxy”, used alone or as part of another group, means the radical —OR, where R is an alkyl group as defined herein.

As used herein, the terms “halo,” “halogen,” and “halide” refer to any suitable halogen, including —F, —Cl, —Br, and —I.

As used herein, the term “mercapto” refers to an —SH group.

As used herein, the term “cyano” refers to a —CN group.

As used herein, the term “carboxylic acid” refers to a —C(O)OH group.

As used herein, the term “hydroxyl” refers to an —OH group.

As used herein, the term “nitro” refers to an —NO₂ group.

As used herein, the term “sulfonyl” refers to the SO₂⁻ group. The “sulfonyl” may refer to a sulfonyl group, which is, for example, an alkylsulfonyloxy group such as a methylsulfonyloxy or ethylsulfonyloxy group and an aromatic sulfonyloxy group such as a benzenesulfonyloxy or tosyloxy group.

As used herein, the terms “ether” and “alkylether” are represented by the formula Rₐ—O—R_{b}, where Rₐ and R_{b} can be, independently, an alkyl, cycloalkyl, alkenyl, cycloalkenyl, alkynyl, cycloalkynyl, aryl, or heteroaryl group as described herein. The term “polyether” as used herein is represented by the formula —(Rₐ—O—R_{b})ₓ—, where Rₐ and R_{b} can be, independently, an alkyl, cycloalkyl, alkenyl, cycloalkenyl, alkynyl, cycloalkynyl, aryl, or heteroaryl group described herein and “x” is an integer from 1 to 500. Examples of polyether groups include polyethylene oxide, polypropylene oxide, and polybutylene oxide.

As used herein, the term “acyl”, used alone or as part of another group, refers to a —C(O)R radical, where R is any suitable substituent such as aryl, alkyl, alkenyl, alkynyl, cycloalkyl or other suitable substituent as described herein.

As used herein, the terms “alkylthio” and “thiyl,” used alone or as part of another group, refers to an alkyl group, as defined herein, appended to the parent molecular moiety through a thio moiety, as defined herein. Representative examples of alkylthio include, but are not limited to, methylthio, ethylthio, tert-butylthio, hexylthio, and the like.

As used herein, the term “amino” means the radical —NH₂.

As used herein, the term “alkylamino” or “mono-substituted amino”, used alone or as part of another group, means the radical —NHR, where R is an alkyl group.

As used herein, the term “disubstituted amino”, used alone or as part of another group, means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, and heterocycloalkyl.

As used herein, the term “ester”, used alone or as part of another group, refers to a —C(O)OR radical, where R is any suitable substituent such as alkyl, cycloalkyl, alkenyl, alkynyl or aryl.

As used herein, the term “alkynyl”, used alone or as part of another group, refers to unsaturated linear or branched hydrocarbon radical that contains one triple bond. The hydrocarbon radical contains at least 2 carbon atoms, but preferably contains 3 to 20 carbon atoms.

As used herein, the term “heterocycloalkyl”, used alone or as part of another group, refers to a non-aromatic monocyclic or polycyclic ring comprising carbon and hydrogen atoms and at least one heteroatom, preferably, 1 to 4 heteroatoms selected from nitrogen, oxygen, and sulfur. A heterocycloalkyl group can have one or more carbon-carbon double bonds or carbon-heteroatoms double bonds in the ring as long as the ring is not rendered aromatic by their presence. Examples of heterocycloalkyl groups include aziridinyl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 2 to 6 carbon atoms and from 1 to 3 heteroatoms, referred to herein as (C.sub.1-C.sub.6) heterocycloalkyl.

As used herein, the term “cycloalkenyl” used alone or as part of another group, refers to a hydrocarbon moiety containing a ring of carbon atoms and one or more double bonds in the cycle that do not form an aromatic ring. The ring of carbon atoms can contain about 3 to 12 carbon atoms, or about 3 to 10 carbon atoms, or about 3 to 8 carbon atoms, or about 3 to 6 carbon atoms.

As used herein, the term “amide”, used alone or as part of another group, refers to a —C(O)NRₐR_{b} radical, where Rₐ and R_{b} are any suitable substituent such as alkyl, cycloalkyl, alkenyl, alkynyl or aryl.

As used herein, the term “haloalkyl”, used alone or as part of another group, means an alkyl group that is substituted with one or more fluorine, chlorine, bromine or iodine atoms. Haloalkyl groups include, for example, fluoromethyl, difluoromethyl, trifluoromethyl, pentafluoroethyl, 1,1-difluoroethyl, chloromethyl, chlorofluoromethyl and trichloromethyl groups.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH—CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

As used herein, the term “ether” is represented by the formula Ra-O—Rb, where Ra and Rb can be, independently, an alkyl, cycloalkyl, alkenyl, cycloalkenyl, alkynyl, cycloalkynyl, aryl, or heteroaryl group as described herein. The term “polyether” as used herein is represented by the formula —(Ra-O—Ra)x-, where Rₐ and Ra can be, independently, an alkyl, cycloalkyl, alkenyl, cycloalkenyl, alkynyl, cycloalkynyl, aryl, or heteroaryl group described herein and “x” is an integer of from 1 to 500. Examples of polyether groups include polyethylene oxide, polypropylene oxide, and polybutylene oxide.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

As used herein, the term “unsubstituted” refers to a moiety (such as an alkyl group) that is not bonded to one or more additional organic or inorganic substituent radical as described above, meaning that such a moiety is only substituted with hydrogens.

### B. Carbonylative Hydrofunctionalization Methods

The methods disclosed herein are directed to carbonylative hydrofunctionalizations of alkenes (also referred to as olefin) using a simple, inexpensive cobalt catalyst under mild conditions and using light (e.g., visible light) to produce amides, amines, esters, carboxylic acids, ethers, alcohols, or aldehydes. This type of carbonylative coupling was surprising and unexpected, as such a transformation under these mild reaction conditions has not been observed before. A general reaction scheme of this transformation is shown in Scheme 3 below.

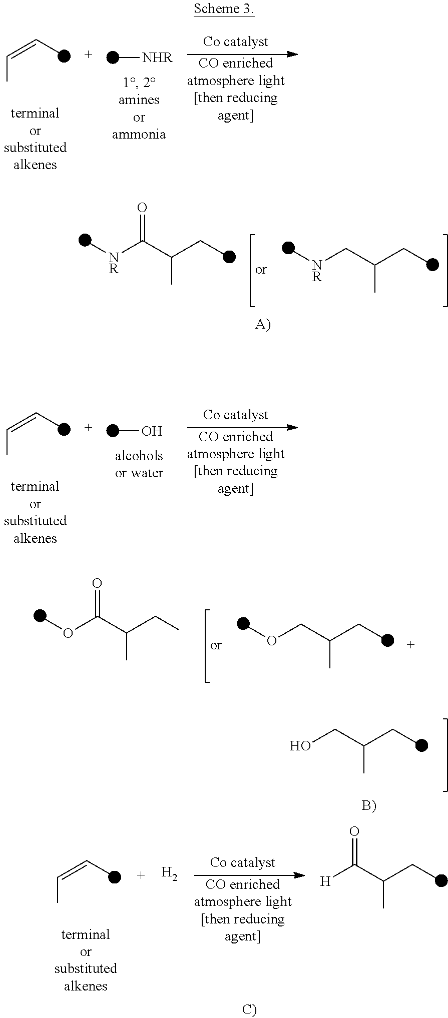

### General Transformations Accessible Via Carbonylative Hydrofunctionalization

According to Scheme 3, the methods disclosed herein produce either amides or amines via carbonylative couplings with nucleophilic substrates such as amines (e.g., alkylamines or ammonia; Scheme 3A), esters, ethers or alcohols with alcohol nucleophiles (Scheme 3B), or aldehydes with hydrogen (Scheme 3C). Additional nucleophilic substrates that can be used in the disclosed carbonylative coupling include water and thiols. For the alkene, it can either be a terminal alkene or a substituted alkene. For substituted alkenes (particularly internal alkenes) chain walking of the double bond can be observed to render a terminal alkene at elevated temperatures. The types of products for such alkenes (i.e., branched versus linear formed products) and their corresponding ratio can be modulated by changing the reaction temperature accordingly. Thus, in some embodiments, the ratio of linear products to branched products can be modulated by the type of olefin (i.e., its substitution pattern and/or substituents) and/or the reaction temperature as well as the type of nucleophilic amine (and its substituents and/or substitution pattern).

The amount of nucleophilic substrate to olefin can vary. In some embodiments, the molar ratio of nucleophilic substrate to olefin ranges from about 1:12 to about 12:1, from about 1:10 to about 10:1, from about 1:8 to about 8:1, from about 1:5 to about 5:1, from about 1:3 to about 3:1, from about 1:2 to about 2:1, or is about 1:1.

With respect to the cobalt catalyst, studies of visible light-promoted aminocarbonylation involving simple Co₂(CO)₈ as precatalyst are consistent with the generation of anionic [Co(CO)₃]⁻ and HCo(CO)₃ catalyst species under mild conditions upon visible light irradiation. Thus, the presence of light is essential for the cobalt catalyst to work.

Not to be bound by theory, but it is believed that if HCo(CO)₃ is generated in the presence of alkenes—and this species is stable under the reaction conditions in the presence of Lewis basic nucleophiles, as HCo(CO)₄ is a strongly acidic metal hydride—that simple, inexpensive Co₂(CO)₈ can enable the carbonylative hydrofunctionalization under mild reaction conditions using visible light (Scheme 4).

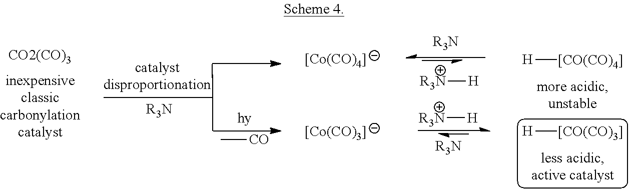

### Light-Promoted Dissociation of CO Generating the Active Metal Hydride Catalyst in the Presence of Lewis Basic Alkylamines

In addition to the above described mild reactions conditions required for the carbonylative hydrofunctionalization methods disclosed herein, the methods also exhibit various other benefits, including: a) the utilization of inexpensive and easily commercially available cobalt catalyst; b) proceeding efficiently with low catalyst loadings; c) proceeding efficiently with low CO pressured atmosphere; d) exhibiting high conversion; e) requiring minimal to no product purification; f) tolerating broad structural diversity in starting materials (e.g., alkene, amine and alcohol building blocks); g) exhibiting minimal to no poisoning of the cobalt catalyst by the formed products (e.g., amines); h) exhibiting high chemo- and regio-selectivity; and i) being able to generate simple and complex molecules more readily than current synthetic methods.

Thus, one aspect of the disclosure is directed to methods of making an amide, the methods comprising contacting an olefin with an amine (i.e., nucleophilic substrate) in the presence of a cobalt catalyst in a carbon monoxide atmosphere while being exposed to light. In some embodiments, the contacting step of the method comprises a reaction mixture containing the amine, the olefin, and the cobalt catalyst. In some embodiments, the reaction mixture further comprises a solvent. The type of amine used in this method can vary. In some embodiments, the amine is a primary amine. In some embodiments, the amine is a secondary amine. In some embodiments, the nucleophile is ammonia. In some embodiments, the olefin is a terminal olefin. In some embodiments, the olefin is a substituted olefin. The various types of amines and/or olefins that can be used in the methods disclosed herein are discussed in more detail below.

Another aspect of the disclosure relates to a method of making an ester, the methods comprising: contacting an olefin with an alcohol (i.e., nucleophilic substrate) in the presence of a cobalt catalyst in a carbon-monoxide atmosphere while being exposed to light. In some embodiments, the alcohol is a primary alcohol. In some embodiments, the alcohol is a secondary alcohol. In some embodiments, the alcohol is a tertiary alcohol. In some embodiments, the nucleophile is water. In some embodiments, the contacting step may further comprise the addition of an additive. The various types of alcohols and additives that can be used in the methods disclosed herein are discussed in more detail below.

Another aspect of the disclosure relates to a method of making a carboxylic acid, the methods comprising: contacting an olefin with water (i.e., nucleophilic substrate) in the presence of a cobalt catalyst in a carbon-monoxide atmosphere while being exposed to light.

Another aspect of the disclosure relates to a method of making an aldehyde, the methods comprising: contacting an olefin with hydrogen in the presence of a cobalt catalyst in a carbon-monoxide atmosphere while being exposed to light. In such embodiments, the hydrogen is hydrogen gas that is mixed with carbon monoxide, e.g., syn gas. In such embodiments, the relative ratio of hydrogen gas to carbon monoxide gas can vary. In some embodiments, the ratio of hydrogen gas to carbon monoxide gas ranges from about 1:10 to about 10:1, from about 5:1 to about 1:5, from about 1:3 to about 3:1, from about 2:1 to about 1:2, or is about 1:1 based on total volume of gas.

As already mentioned above, the contacting step in the methods described above occur under mild reaction condition, such as lower temperature and CO pressure, using light and a cobalt catalyst. A skilled artisan would generally be aware that the reaction conditions (i.e., temperature, CO atmosphere, light, and/or solvent) of the methods disclosed herein can vary as they are dependent upon the type of amine or alcohol being used in combination with the type of olefin (or alkene) being used.

For example, in some embodiments, the temperature used in the contacting step of the disclosed methods can vary. In some embodiments, the temperature is room temperature (e.g., 25° C.). In some embodiments, the temperature is elevated, e.g., higher than room temperature. In some embodiments, the temperature ranges from about 25° C. to about 200° C., from about 30° C. to about 175° C., from about 40° C. to about 160° C., from about 45° C. to about 155° C., from about 50° C. to about 150° C., from about 50° C. to about 140° C., from about 50° C. to about 130° C., from about 50° C. to about 125° C., from about 50° C. to about 120° C., from about 50° C. to about 115° C., from about 50° C. to about 110° C., from about 60° C. to about 100° C., from about 70° C. to about 100° C., from about 80° C. to about 100° C., or from about 85° C. to about 95° C. In some embodiments, the temperature is at least about 20° C., about 30° C., about 40° C., about 50° C., about 60° C., about 70° C., about 75° C., about 80° C., about 85° C., about 90° C., at least about 100° C., or at least about 110° C. In some embodiments, the temperature is about 90° C.

In some embodiments, the concentration of carbon monoxide in the reaction atmosphere can vary. As already mentioned above, the contacting step of the disclosed methods is carried out in a reaction atmosphere enriched with carbon monoxide (also referred to as carbon monoxide atmosphere). The concentration of carbon monoxide in the reaction atmosphere can therefore vary. In some embodiments, carbon monoxide is present in the reaction atmosphere from about 0.1 atm to about 10 atm, from about 0.15 atm to about 8 atm, from about 0.2 atm to about 6 atm, from about 0.35 atm to about 4.5 atm, from about 0.25 atm to about 3 atm, from about 0.5 atm to about 2.5 atm, from about 0.75 atm to about 2.25 atm, from about 0.8 atm to about 1.8 atm, from about 0.85 atm to about 1.5 atm, from about 0.9 atm to about 1.23 atm, or from about 0.95 atm to about 1.15 atm. In some embodiments, carbon monoxide is present in the reaction atmosphere from about 1 atm to about 2 atm, from about 1 atm to about 1.5 atm, or from about 1 atm to about 1.2 atm. In some embodiments, carbon monoxide is present in the reaction atmosphere is less than about 10 atm, about 9 atm, about 8 atm, about 7 atm, about 6 atm, about 5 atm, about 4 atm, about 3 atm, about 2.5 atm, about 2.25 atm, about 2.1 atm, about 2.0 atm, about 1.8 atm, about 1.6 atm, about 1.4 atm, about 1.3 atm, about 1.2 atm, about 1.1 atm, about 1.0 atm, about 0.9 atm, about 0.8 atm, about 0.7 atm, about 0.6 atm, about 0.5 atm, about 0.4 atm, about 0.3 atm, or about 0.2 atm, with a minimum of about 0.1 atm carbon monoxide. In some embodiments, where reaction is carried out in the presence of another gas, e.g., hydrogen, or is a mixed gas, e.g., syngas, the reaction atmosphere can exhibit the same ranges of concentrations as mentioned above for a carbon monoxide atmosphere.

In some embodiments, the type of light used in the contacting step of the methods disclosed herein can vary. In some embodiments, the light used is visible light or ultraviolet light. Thus, in some embodiments, the wavelength of the light used in the contacting step for the disclosed methods varies. For example, in some embodiments, the wavelength is in the ultraviolet region ranging from about 315 nm to about 400 nm, from about 335 nm to about 400 nm, from about 350 nm to about 400 nm, from about 360 nm to about 400 nm, from about 370 nm to about 400, from about 380 nm to about 400, or from about 390 to about 400 nm. In some embodiments, the wavelength is in the visible region from about 400 nm to about 700 nm, from about 400 nm to about 650 nm, from about 400 nm to about 600 nm, from about 400 nm to about 550 nm, from about 400 nm to about 500 nm, from about 400 nm to about 450 nm, or from about 400 nm to about 425 nm. In some embodiments, the wavelength of the light used in the contacting step for the disclosed methods ranges from about 350 nm to about 490 nm, or from about 370 to about 460 nm. In some embodiments, the wavelength ranges from about 350 to about 400 nm, from about 370 to about 410, from about 400 to about 460 nm, or from about 410 nm to about 490 nm. In some embodiments, the wavelength is selected from the group consisting of about 370 nm, about 390 nm, 427 nm, and 440 nm.

The light source used to generate the light employed in the disclosed methods can be any light source generally known in the art to produce visible or ultraviolet light. In some embodiments, the light source is provided by natural sources, i.e., sunlight, whereas in other embodiments, the light source is provided by an artificial source. Exemplary artificial light sources include, but should not be limited to, black light, short-wave ultraviolet lamps, incandescent lamps, gas-discharge lamps, ultraviolet or visible light-emitting diodes (LEDs), and/or ultraviolet lasers.

As already mentioned above, the methods disclosed herein can be carried out without a solvent. Thus, in some embodiments, the contacting step of the methods disclosed herein is carried out neat.

In some embodiments, the contacting step of the methods disclosed herein is carried out in the presence of a solvent. Exemplary solvents include, but are not limited to, methyl tert-butyl ether (MTBE), tert-amyl methyl ether (TAME), tert-hexyl methyl ether (THEME), ethyl tert-butyl ether (ETBE), tert-amyl ethyl ether (TAEE), diisopropyl ether (DIPE), tetrahydrofuran (THF), toluene, tert-amyl alcohol (t-AmOH), isopropylalcohol (iPrOH), diisopropylamine, pivalonitrile, acetonitrile, ethyl acetate (EtOAc), acetone, ethanol (EtOH), methanol (MeOH), trifluoroethanol, N-methyl-2-pyrrolidone (NMP), isopropyl acetate (iPrOAc), and a combination thereof. In some embodiments, the solvent is MTBE.

The amount of solvent, if present, employed in the disclosed methods can vary. For example, in some embodiments, the solvent is present in an amount of from about 0.1% to about 99%, from about 0.5% to about 95%, from about 1% to about 94% from about 5% to about 90%, from about 5% to about 80%, from about 10% to about 70%, from about 15% to about 60%, from about 20% to about 55%, from about 30% to about 50%, or from about 35% to about 45% by weight based on the total weight of the reaction mixture. In some embodiments, the solvent is present in an amount of from about 1% to 75%, from about 1% to about 70% from about 1% to about 60%, from about 1% to about 50%, from about 1% to about 40%, from about 1% to about 30%, from about 1% to about 20%, or from about 1% to about 10% by weight based on the total weight of the reaction mixture.

Another aspect of the current disclosure are methods of making amines, ethers, or alcohols from the corresponding amides and esters obtained from the above described method, respectively. This can be accomplished by adding a reducing agent to the amide or ester generated by the above described methods.

Thus, in some embodiments, a method of making an amine as disclosed herein comprises obtaining an amide prepared by the hydroaminocarbonylation method disclosed herein and then contacting such an amide with a reducing agent.

In some embodiments, a method of making an ether as disclosed herein comprises obtaining an ester prepared according to the methods disclosed herein and then contacting such an ester with a reducing agent.

In some embodiments, a method of making an alcohol as disclosed herein comprises obtaining an ester prepared according to the methods disclosed herein and then contacting such an ester with a reducing agent.

In some embodiments, the reducing agent is a silane-containing reducing agent. Exemplary silane-containing reducing agents include, but are not limited to, trimethoxysilane, tripropylsilane, dimethoxy(methyl) silane, phenyl silane, triethoxysilane, diphenyl silane, or combinations thereof. In some embodiments, the reducing agent is trimethoxysilane. In some embodiments, the reducing agent is phenyl silane or diphenyl silane.

The amount of reducing agent employed in this method can vary. In some embodiments, the reducing agent is present in an amount of from about 0.1% to about 99%, from about 1% to about 90%, from about 10% to about 80%, from about 20% to about 70%, from about 30% to about 60%, or from about 40% to about 50% by weight based on the total weight of the reaction mixture (which contains the amide or ester and/or solvent).

In some embodiments, the reducing agent is present in an amount of from about 0.1 to about 5.0 equivalence (equiv.), from about 0.5 to about 4.0 equiv., from about 0.75 to about 3.0 equiv., from about 1.0 to about 3.0 equiv., from about 1.5 to about 3.0 equiv. from about 2.0 to about 3.0 equiv., or from about 2.25 to about 2.75 equiv. based on the amount of olefin present (i.e., 1 equiv.). In some embodiments, the amount of reducing agent ranges from 2.5 to 3.0 equiv. based on the amount of olefin present (i.e., 1 equiv.).

In some embodiments, the product (e.g., amide, ester, amine, ether, aldehyde, acid and/or alcohol) can be obtained in a yield of at least about 50%, about 55%, about 60%, about 65%, about 70%, about 75%, about 80%, about 85%, about 90%, or at least about 95%.

Another aspect of the disclosure is directed towards methods carbonylative hydrofunctionalizations of alkynes using a simple, inexpensive cobalt catalyst under mild conditions and using light (e.g., visible light) to produce acrylamides and acrylates. Such a method comprises contacting an alkyne with an amine or alcohol (i.e., nucleophilic substrate) in the presence of a cobalt catalyst in a carbon monoxide atmosphere while being exposed to light. In some embodiments, the contacting step of the method comprises a reaction mixture containing the amine or alcohol, the alkyne, and the cobalt catalyst. In some embodiments, the reaction mixture further comprises a solvent. The type of amine or alcohol used in this method can vary. In some embodiments, the amine or alcohol is a primary amine or alcohol. In some embodiments, the amine or alcohol is a secondary amine or alcohol. In some embodiments, the alkyne is a terminal alkyne. The various types of amines, alcohols and/or alkynes that can be used in the methods disclosed herein are discussed in more detail below. For Example, Scheme 5 below shows the methods disclosed herein that produce acrylamides via carbonylative couplings with alkynes and nucleophilic amines (e.g., alkylamines or ammonia; Scheme 5A), or acrylates via carbonylative couplings with alkynes and alcohol nucleophiles (Scheme 5B).

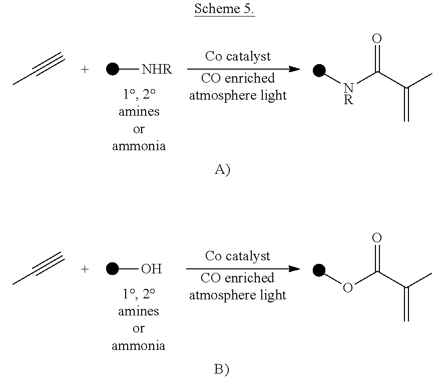

### General Transformations Accessible Via Carbonylative Hydrofunctionalization

In addition, the methods disclosed herein can also extend to the preparation of cyclic amides and esters (lactams and lactones, respectively) as is shown in the general scheme below (Scheme 13).

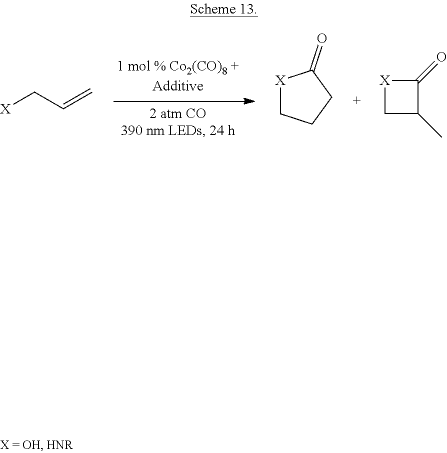

### Cobalt-Catalyzed Carbonylative Hydrofunctionalizations for Preparing Cyclic Amides and Esters; n=1-10

In such a method, the method comprises contacting a functionalized alkene, which contains a terminal alcohol (—OH) or amine group (—NH₂) with a cobalt catalyst in a carbon monoxide atmosphere while being exposed to light. In other words, the functionalized alkenes serves as two reaction partners in this method: the alkene and the nucleophilic substrate. In some embodiments, the reaction mixture further comprises a solvent. In such a method, the functionalized alkene is able to react with itself, intramolecularly, to form the cyclic amide or ester.

A skilled artisan would be able to adjust and optimize the above reaction parameters to obtain the most efficient reaction conditions for the hydroaminocarbonylation. A skilled artisan would know that the above reaction parameters are dependent on the reaction materials being used, i.e., the type of nucleophilic substrate (e.g., amine and/or alcohol) and/or the type of olefin being used.

Thus, the methods disclosed herein generally comprise an alkene or alkyne, optionally a nucleophilic substrate (e.g., an amine or alcohol), a cobalt catalyst, a carbon monoxide atmosphere and light. For methods comprising an alkene and an amine as a nucleophilic substrate amides are generated as products. For methods comprising an alkene and an alcohol as a nucleophilic substrate esters are generated as products. For methods comprising an alkene and water as a nucleophilic substrate carboxylic acids are generated as products. Amide and ester products can be further modified with reducing agents to afford the corresponding amines or ethers. For methods comprising an alkene that is functionalized to also include a hydroxy group (—OH) or amine (—NH₂), no nucleophilic substrate is present as the alkene already contains the required functional group of a nucleophilic substrate and can react with itself to form cyclic amides and esters. Thus, in those methods where such functionalized alkenes are present, the nucleophilic substrate may be absent.

For methods comprising an alkyne and an amine as a nucleophilic substrate acrylamides are generated as products. For methods comprising an alkyne and an alcohol as a nucleophilic substrate acrylates are generated as products.

Additional methods disclosed herein are directed towards replacing the nucleophilic substrate with hydrogen. Such methods would comprise an alkene, hydrogen, a cobalt catalyst, a carbon monoxide atmosphere and light to produce aldehydes.

In any of the above methods, an additive may further be added. These reaction materials are disclosed in more detail below.

### C. Nucleophilic Substrate

The methods disclosed herein can comprise a nucleophilic substrate such as an amine, water or an alcohol, although the disclosed methods should not be limited thereto. As mentioned above, in some embodiments, the methods disclosed herein may contain hydrogen instead of a nucleophilic substrate. In some embodiments, the method disclosed herein may contain no nucleophilic substrate provided that the alkene is properly functionalized as described above. Additional nucleophilic substrates that can be used in the disclosed methods include thiols and/or hydrides.

In some embodiments, the nucleophilic substrate used in the methods disclosed herein is a compound of Formula I:

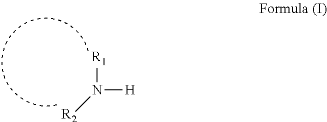

wherein R₁ and R₂ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₘaryl, and —(CH₂)ₚheteroaryl, wherein m and p are integers independently selected from about 1 to about 20 atoms; and
wherein [symbol] represents a single bond, if present, between R₁ and R₂.

In some embodiments, R₁ and/or R₂ are substituted with one or more substituents as described above.

In some embodiments, m and p are integers independently selected from about 1 to about 10 atoms, from about 1 to about 5 atoms, or from about 1 to about 3 atoms. In some embodiments, m is 1. In some embodiments, p is 2.

In some embodiments, R₁ and R₂ are independently selected from the group consisting of hydrogen, —(C₁-C₆)alkyl, —(C₃-C₆)cycloalkyl, —(CH₂)ₘaryl, and —(CH₂)ₚheteroaryl.

In some embodiments, the alkyl group is linear. In some embodiments, the alkyl group is branched. In some embodiments, R₁ and R₂ are independently selected from the group consisting of hydrogen, —(CH₂)₅CH₃ and —C(CH₃)₃. In some embodiments, R₁ is hydrogen and R₂ is —(CH₂)₅CH₃ or —C(CH₃)₃.

In some embodiments, R₁ and R₂ are both hydrogen. For example, in some embodiments, the amine is ammonia.

In some embodiments, R₁ and R₂ are independently selected from the group consisting of hydrogen, and (C₃-C₆)cycloalkyl. In some embodiments, R₁ is hydrogen and R₂ is —(C₃-C₆)cycloalkyl. In some embodiments, R₁ is hydrogen and R₂ is cyclopropyl.

In some embodiments, R₁ and R₂ are independently selected from the group consisting of hydrogen, —(C₁-C₆)alkyl, —(CH₂)ₘaryl, and —(CH₂)ₚheteroaryl. In some embodiments, R₁ is hydrogen and R₂ is —(CH₂)ₚheteroaryl. In some embodiments, R₁ is —(C₁-C₆)alkyl and R₂ is —(CH₂)ₘaryl. In some embodiments, the amine is selected from the group consisting of

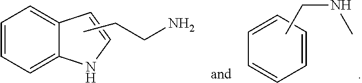

In some embodiments, a single bond is present between R₁ and R₂ in the compound of Formula (I) to form a heterocycle with at least one heteroatom (i.e., a nitrogen atom). Exemplary heterocycles include, but are not limited to, piperazine, piperdine, and morpholine.

In some embodiments, the amine is a compound selected from the groups consisting of

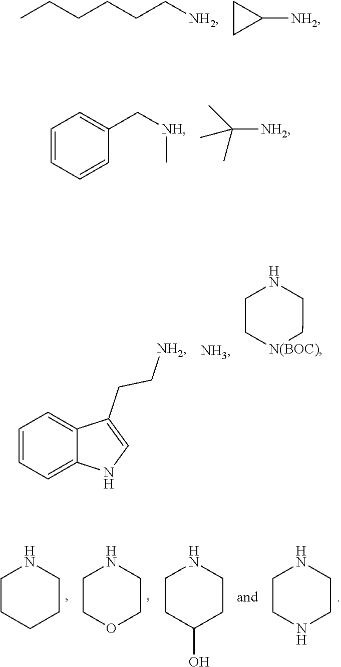

Another aspect of the current disclosure relates to the use of an alcohol as a nucleophilic substrate in the disclosed methods to prepare esters. In some embodiments, the alcohol is a primary alcohol. In some embodiments, the alcohol is a secondary alcohol. In some embodiments, the alcohol employed in the methods disclosed herein is a compound of Formula Ia:

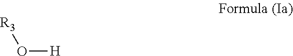

wherein R₃ is selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₘaryl, and —(CH₂)ₚheteroaryl, wherein m and p are integers independently selected from about 1 to about 20 atoms.

In some embodiments, R₃ is substituted with one or more substituents as described above.

In some embodiments, R₃ is selected from the group consisting of hydrogen, —(C₁-C₆)alkyl, —(C₃-C₆)cycloalkyl, —(CH₂)ₘaryl, and —(CH₂)ₚheteroaryl.

In some embodiments, the alkyl group is linear. In some embodiments, the alkyl group is branched. In some embodiments, R₃ is methyl. In some embodiments, the alcohol is methanol.

In some embodiments, the nucleophilic substrate is water.

The amount of the nucleophilic substrate (i.e., amine, water or alcohol) used in the disclosed methods can vary. In some embodiments, the amount of nucleophilic substrate present in the methods disclosed herein ranges from about 1% to about 99%, from about 10% to about 90%, from about 15% to about 85%, from about 20% to about 80%, from about 25% to about 75%, from about 30% to about 70%, from about 35% to about 65%, from about 40% to about 60%, or from about 45% to about 55% by weight based on the total weight of the reaction mixture. It would be understood by a skilled artisan that the described nucleophilic substrates above are exemplary starting materials that can be used in the disclosed methods and is not meant to limit the scope of the methods in any way. It would be apparent to a skilled artisan that other nucleophilic substrates would be suitable for the methods disclosed herein.

### D. Olefin and Alkynes

The methods disclosed herein comprise an olefin (also referred to as an alkene) or an alkyne as a starting material for the disclosed hydrofunctionalization reactions. In some embodiments, the olefin is a terminal olefin. In some embodiments, the olefin is a substituted olefin. In some embodiments, the olefin is a mono-substituted olefin. In some embodiments, the olefin is a di-substituted olefin. In some embodiments, the olefin is a tri-substituted olefin. In some embodiments, the olefin is a tetra-substituted olefin. In some embodiments, the olefin is a non-cyclic olefin. In some embodiments, the olefin is a cyclic olefin. In some embodiments, the olefin contains one double bond. In some embodiments, the olefin contains more than one double bond. In some embodiments, the olefin is a compound of Formula II:

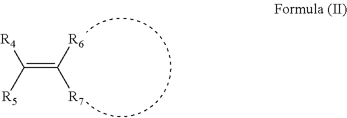

wherein R₄, R₅, R₆ and R₇ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms; and
wherein [symbol] represents a bond, if present, between R₆ and R₇.

In some embodiments, R₄, R₅, R₆ and/or R₇ can be substituted with one or more substituents as described above.

In some embodiments, at least one of R₄-R₇ is hydrogen. In some embodiments, R₄ is hydrogen. In some embodiments, R₄ and R₅ are hydrogen. In some embodiments, R₄, R₅, and R₆ are hydrogen. In some embodiments, R₄ and R₇ are hydrogen.

In some embodiments, R₄ is hydrogen and R₅, R₆, and R₇ are not hydrogen. In some embodiments, R₄ is hydrogen and R₅, R₆ and R₇ are independently selected from the group consisting of alkyl, cycloalkyl, heterocycloalkyl, aryl, and heteroaryl. In some embodiments, R₄ is hydrogen and R₅, R₆ and R₇ are independently selected from —(C₁-C₆)alkyl. In some embodiments, R₄ is hydrogen and a bond is present between the R₆ and R₇ (C₁-C₆)alkyl groups forming a ring. In such embodiments, the ring is a (C₃-C₁₀)cycloalkyl. In a specific embodiment, R₆ and R₇ form a C₆-cycloalkyl.

In some embodiments, R₄ and R₅ are hydrogen and R₆ and R₇ are independently selected from the group consisting of alkyl, cycloalkyl, heterocycloalkyl, aryl, and heteroaryl. In some embodiments, R₄ and R₅ are hydrogen and R₆ and R₇ are independently selected from (C₁-C₆) alkyl. In some embodiments, R₄ and R₅ are hydrogen and a bond is present between the R₆ and R₇ (C₁-C₆)alkyl groups forming a ring. In such embodiments, the ring is a (C₃-C₁₀)cycloalkyl. In a specific embodiment, R₆ and R₇ form a C₆-cycloalkyl.

In some embodiments, R₄, R₅, and R₆ are hydrogen and R₇ is selected from the group consisting of alkyl, cycloalkyl, heterocycloalkyl, aryl, and heteroaryl. In some embodiments, R₇ is a (C₃-C₁₀)cycloalkyl. In some embodiments, R₇ is a C₆-cycloalkyl.

In some embodiments, R₄ and R₇ are hydrogen and R₅ and R₆ are independently selected from the group consisting of alkyl, cycloalkyl, heterocycloalkyl, aryl, and heteroaryl. In some embodiments, R₄ and R₇ are selected from (C₁-C₁₀) alkyl. In some embodiments, R₄ and R₇ are the same (C₁-C₁₀)alkyl group. In some embodiments R₄ and R₇ are different (C₁-C₁₀)alkyl group.

In some embodiments, none of R₄-R₇ is hydrogen. In some embodiments, R₄, R₅, R₆ and R₇ are independently selected from the group consisting of alkyl, cycloalkyl, heterocycloalkyl, aryl, heteroaryl, alkenyl, alkynyl, haloalkyl, heteroalkyl, cycloalkenyl, —(CH₂)ₘaryl, and —(CH₂)ₚheteroaryl, wherein m and p are integers independently selected from about 1 to about 20 atoms. In some embodiments, R₄, R₅, R₆ and R₇ are independently selected from (C₁-C₁₀)alkyl. In some embodiments, R₄, R₅, R₆ and R₇ are methyl.

In some embodiments, R₄ or R₅ is selected from the group consisting of —(CH₂)ₙOH and —(CH₂)_{q}NH₂. In some embodiments, n and q are integers independently selected from the group consisting of 1-10.

Another aspect of the current disclosure relates to the use of an olefin that is a compound of Formula III:

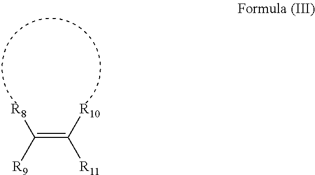

wherein R₈, R₉, R₁₀ and R₁₁ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms; and
wherein [symbol] represents a bond, if present, between R₈ and R₉.

In some embodiments, R₈, R₉, R₁₀ and/or R₁₁ can be substituted with one or more substituents as described above.

In some embodiments, R₉ and R₁₁ are hydrogen. In some embodiments, R₉ and R₁₁ are hydrogen and R₈ and R₁₀ are independently selected from (C₁-C₆)alkyl. In some embodiments, R₉ and R₁₁ are hydrogen and a bond is present between the R₈ and R₁₀ (C₁-C₆)alkyl groups forming a ring. In such embodiments, the ring is a (C₃-C₁₀)cycloalkyl. In a specific embodiment, R₈ and R₉ form a C₆-cycloalkyl.

In some embodiments, R₉ is hydrogen. In some embodiments, R₉ is hydrogen and R₈, R₁₀, and R₁₁ are independently selected from (C₁-C₆)alkyl. In some embodiments, R₉ is hydrogen, R₁₁ is (C₁-C₆)alkyl and a bond is present between the R₈ and R₁₀ (C₁-C₆)alkyl groups forming a ring. In such embodiments, the ring is a (C₃-C₁₀)cycloalkyl. In a specific embodiment, R₈ and R₉ form a C₆-cycloalkyl.

In some embodiments, R₉ or R₁₁ is selected from the group consisting of —(CH₂)ₙOH and —(CH₂)_{q}NH₂. In some embodiments, n and q are integers independently selected from the group consisting of 1-10.

In some embodiments, the olefin is a compound selected from the group consisting of:

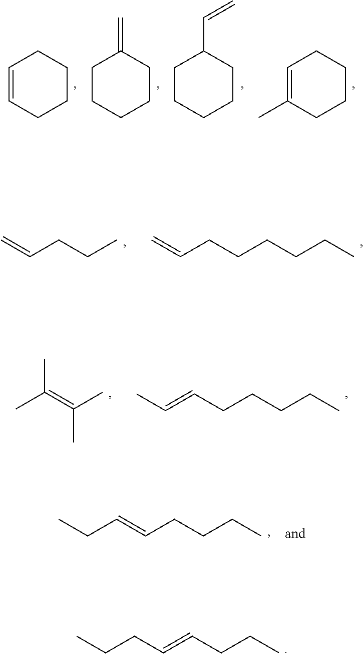

In some embodiments, the starting material for the disclosed hydrofunctionalization reactions is an alkyne. In some embodiments, the alkyne is a an alkyne of Formula VII:

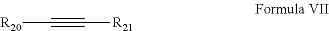

wherein R₂₀ is selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms.

In some embodiments, R₂₀ can be substituted with one or more substituents as described above.

In some embodiments, R₂₀ is (C₁-C₆)alkyl.
wherein R₂₁ is selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms.

In some embodiments, R₂₁ can be substituted with one or more substituents as described above.

In some embodiments, R₂₁ is (C₁-C₆)alkyl.

The amount of olefin or alkyne used in the methods disclosed herein can vary. In some embodiments, the amount of olefin or alkyne present in the methods disclosed herein ranges from about 1% to about 99%, from about 10% to about 90%, from about 15% to about 85%, from about 20% to about 80%, from about 25% to about 75%, from about 30% to about 70%, from about 35% to about 65%, from about 40% to about 60%, or from about 45% to about 55% by weight based on the total weight of the reaction mixture.

### E. Cobalt Catalyst

The carbonylative hydrofunctionalization methods disclosed employ a cobalt catalyst. In some embodiments, the cobalt catalyst is selected from a group consisting of dicobalt octacarbonyl Co₂(CO)₈, sodium tetracarbonylcobaltate (NaCo(CO)₄), and potassium tetraacarbonylcobaltate (KCo(CO)₄). In some embodiments, the cobalt catalyst employed in the disclosed methods is dicobalt octacarbonyl (CAS No. 10210-68-1; cobalt carbonyl). Dicobalt octacarbonyl is an organocobalt compound, wherein each molecule consists of two cobalt atoms bound to eight carbon monoxide ligands. Although multiple structural isomers are known, the general structure of Co₂(CO)₈ is typically illustrated as:

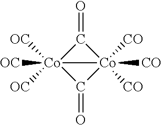

Dicobalt octacarbonyl is used in various applications but it best known for its use as a catalyst for hydroformylation—the conversion of alkenes to aldehydes. Such hydroformylations include active catalyst species, such as cobalt tetracarbonyl hydride H[Co(CO)₄], which are responsible for catalyzing these hydroformylations. Since HCo(CO)₄ decomposes so readily, it is usually generated in situ.

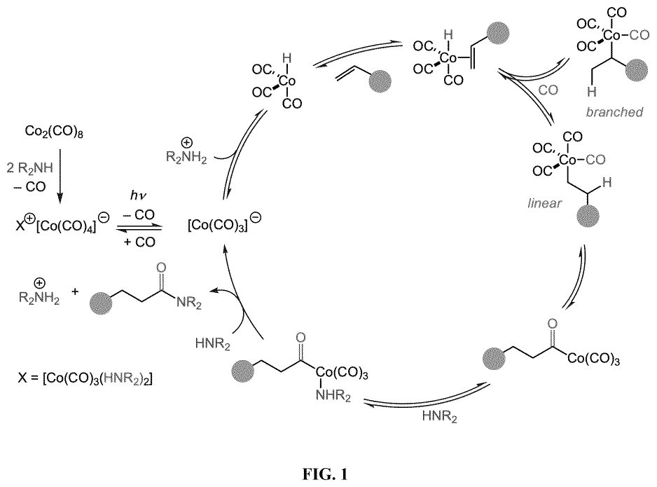

The hydroaminocarbonylation methods disclosed herein employ cobalt catalysts such as Co₂(CO)₈ from which tetracarbonyl hydride H [Co(CO)₄] can be derived from. A possible catalytic mechanism of the hydroaminocarbonylation disclosed herein is shown in FIG. 1. Tetracarbonyl hydride H[Co(CO)₄] is produced and is disproportionated with light promoted loss of a CO ligand thereby generating the cobalt tricarbonyl anion which is subsequently protonated yielding HCo(CO)₃. Alkene coordination is then followed by hydrocobaltation, leading to regioisomeric alkylcobalt intermediates which are capable of isomerization via sequential β-hydride elimination/reinsertion processes to ultimately deliver the terminal alkylcobalt with acyclic substrates. Migratory insertion of a CO ligand then takes place, furnishing an acylcobalt tricarbonyl. The amine nucleophile then coordinates to the metal center, followed by a concerted addition/reductive elimination to produce the protonated amide product and the cobalt tricarbonyl anion, which undergo proton exchange to regenerate HCo(CO)₃. This unique catalytic mode of simple cobalt carbonyl enables the hydroaminocarbonylation which provides an efficient, general, and highly atom—economical approach to amides and amines from fundamental chemical building blocks.

The amount of cobalt catalyst used in the disclosed hydroaminocarbonylation can vary. In some embodiments, the amount of cobalt catalyst is from about 0.1 mol % to about 5 mol %, from about 0.25 mol % to about 4.5 mol %, from about 0.5 mol % to about 4.0 mol %, from about 0.5 mol % to about 3.5 mol %, from about 0.5 mol % to about 3 mol %, from about 0.5 mol % to about 2.5 mol %, from about 0.5 mol % to about 2 mol %, from about 0.5 mol % to about 1.5 mol %, or from about 0.75 mol % to about 1.25 mol %. In some embodiments, the amount of cobalt-containing catalyst is at least about 0.1 mol %, 0.25 mol %, 0.3 mol %, 0.4 mol %, 0.5 mol %, 0.6 mol %, 0.75 mol %, 0.8 mol %, 0.9 mol %, 1.0 mol %, 1.25 mol %, 1.5 mol %, or at least 2 mol %.

### F. Additive

The methods disclosed herein may comprise an additive. In some embodiments, the additive is an amine and/or acid to facilitate the generation of the active catalyst, although the disclosed methods should not be limited thereto. In some embodiments, the additive used in the methods disclosed herein is a compound of Formula I, IV, V, VI, VII, or any combination thereof. In some embodiments, the additive used in the methods disclosed herein is a compound of Formula I.

In some embodiments, the additive used in the methods disclosed herein is a compound of Formula IV:

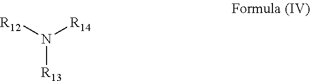

wherein R₁₂, R₁₃ and R₁₄ are independently selected from the group consisting of alkyl, cycloalkyl, heterocycloalkyl, aryl, heteroaryl, alkenyl, alkynyl, haloalkyl, heteroalkyl, cycloalkenyl, —(CH₂)ₘaryl, and —(CH₂)ₚheteroaryl, wherein m and p are integers independently selected from about 1 to about 20 atoms.

In some embodiments, the additive is a compound selected from the group consisting of:

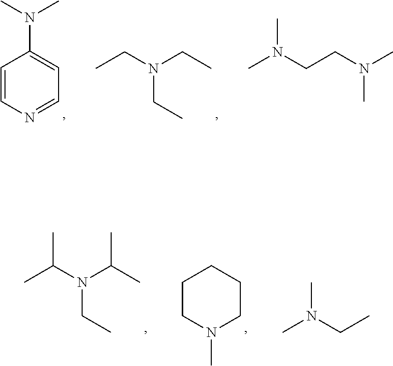

In some embodiments, the additive used in the methods disclosed herein is a compound of Formula V:

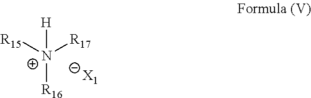

wherein R₁₅, R₁₆ and R₁₇ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₘaryl, and —(CH₂)ₚheteroaryl, wherein m and p are integers independently selected from about 1 to about 20 atoms; and X₁ is independently selected from the group consisting of acetate, chloride and tosylate.

In some embodiments, the additive is a compound selected from the group consisting of:

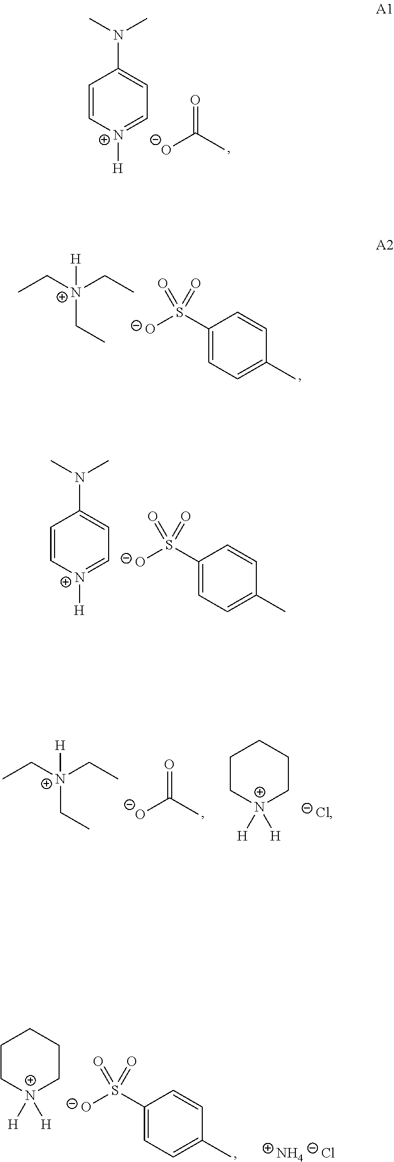

In some embodiments, the additive used in the methods disclosed herein is a compound of Formula VI:

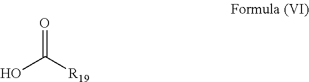

wherein R₁₉ is independently selected from the group consisting of alkyl, cycloalkyl, heterocycloalkyl, aryl, heteroaryl, alkenyl, alkynyl, haloalkyl, heteroalkyl, cycloalkenyl, —(CH₂)ₘaryl, and —(CH₂)ₚheteroaryl, wherein m and p are integers independently selected from about 1 to about 20 atoms.

In some embodiments, the additive is a compound selected from the group of:

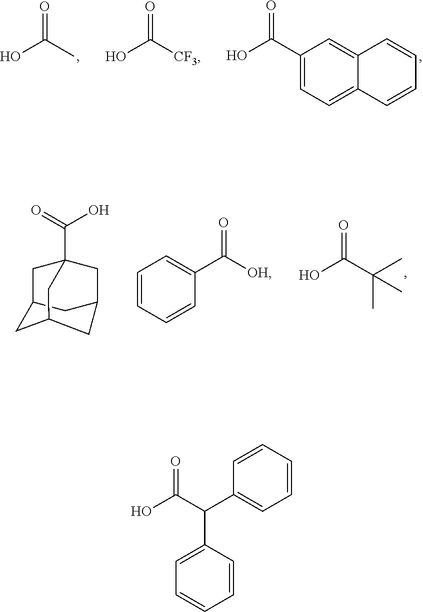

In some embodiments, the additive is para-toluene sulfonic acid.

The amount of the additive used in the disclosed methods can vary. In some embodiments, the amount of additive present in the methods disclosed herein ranges from about 1% to about 99%, from about 10% to about 90%, from about 15% to about 85%, from about 20% to about 80%, from about 25% to about 75%, from about 30% to about 70%, from about 35% to about 65%, from about 40% to about 60%, or from about 45% to about 55% by weight based on the total weight of the reaction mixture.

It would be understood by a skilled artisan that the described additive above are exemplary starting materials that can be used in the disclosed methods and is not meant to limit the scope of the methods in any way. It would be apparent to a skilled artisan that other additives would be suitable for the methods disclosed herein.

### G. Scope of Carbonylative Hydrofunctionalization Methods

The methods disclosed herein are directed to carbonylative hydrofunctionalizations of alkenes (also referred to as olefin) using a simple, inexpensive cobalt catalyst under mild conditions and using light (e.g., visible light) to produce amides, esters, aldehydes, amines, ethers, or alcohols. Shown below are general reaction conditions when using an amine and an olefin as starting materials (Scheme 6).

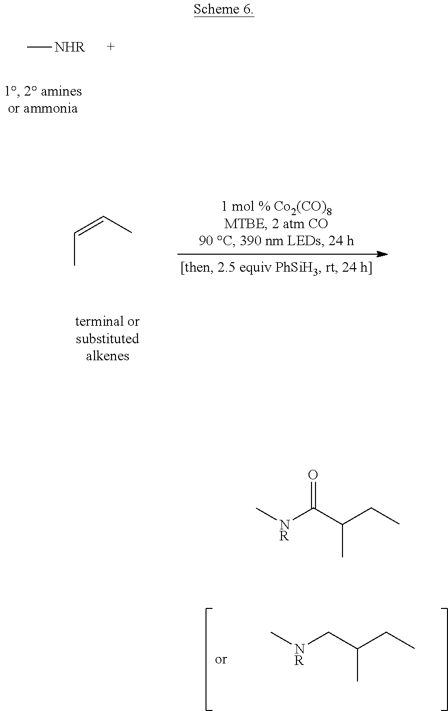

### Cobalt-Catalyzed Hydroaminocarbonylation and Hydroaminomethylation Under Mild Conditions Promoted by Visible Light

The methods disclosed herein show that the simple cobalt carbonyl is a highly effective catalyst for the hydroaminocarbonylation at low catalyst loading and CO pressure upon irradiation with 390 nm purple LEDs. Furthermore, the methods disclosed herein are highly efficient using 1 equiv of both alkene and amine coupling partners. While the reaction can be performed at ambient temperature, heating generally accelerates the reaction. This is particularly noticeable when internal alkenes are used as starting materials. At room temperature, the reaction produces a mixture of branched and linear products, whereas at increasing reaction temperature chain walking of the internal alkene is facilitated thereby changing the ratio of linear vs. branched products (i.e., the amount of linear products increases). Thus, it is possible to control the ratio of linear vs. branched products via the reaction temperature.

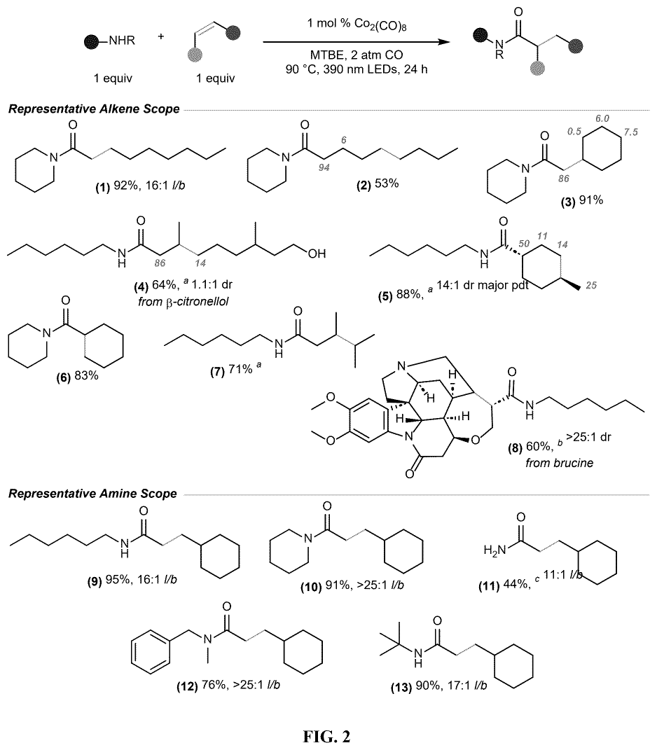

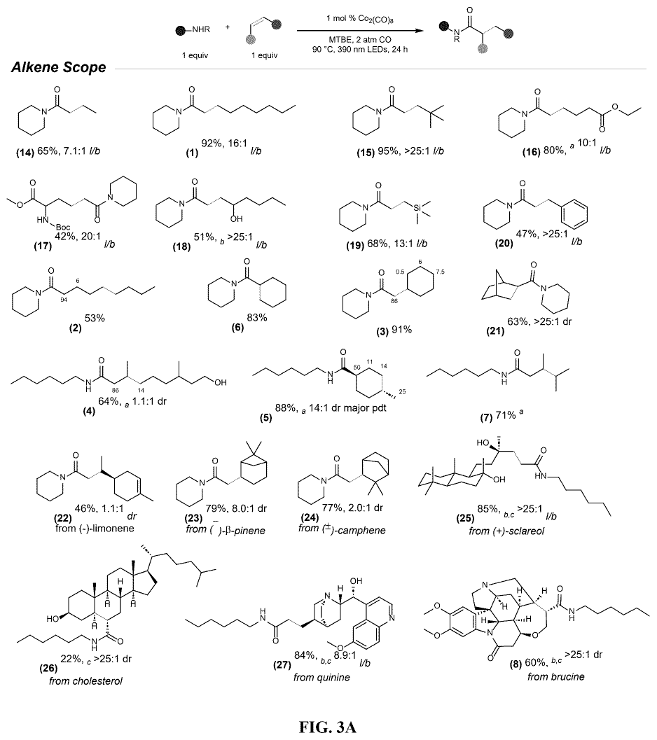

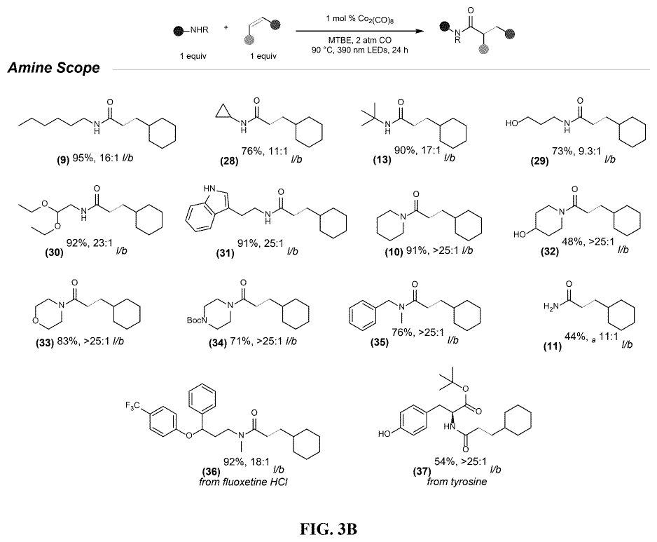

A representative substrate scope of the hydroaminocarbonylation demonstrating the successful coupling of the major classes of alkenes and amines is depicted in FIG. 2. With respect to alkene scope, terminal, 1,1- and 1,2-disubstituted, tri- and tetrasubstituted alkenes are all excellent substrates in the hydroaminocarbonylation. With internal alkene substrates, efficient chain walking can be observed, leading to selective functionalization of the end of the chain (FIG. 2, compounds 2, 4 and 7). A larger list of more diverse alkene substrates are depicted in FIG. 3A.

The scope with respect to the alkylamine coupling partner is equally broad, highlighting the overall utility of the methods. Diverse primary and secondary acyclic and cyclic amines all react efficiently in couplings with vinylcyclohexane (FIG. 2, compounds 9-13). Additionally, the hydroaminocarbonylation with ammonia gas is also successful, as the use of 4.5 equivalents NH₃ in iPrOH provided primary amide 12 in 44% isolated yield. A larger list of more diverse amine substrates are depicted in FIG. 3B.

Next, a reaction using a mixture of all octene isomers as substrate produced amide 1 in similar yield and selectivity to the reaction of 1-octene, demonstrating the regioconvergence of the hydroaminocarbonylation (Scheme 7).

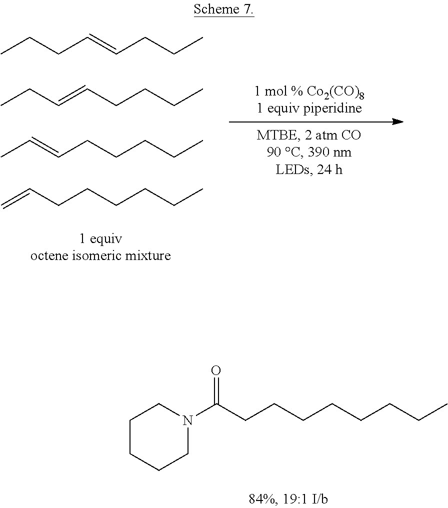

### Hydroaminocarbonylation of a Mixture of Octenes Converges to the Linear Product with High Regioselectivity

The high linear selectivity of the reaction indicates rapid chain walking of intermediate alkylcobalt species. Furthermore, the aminocarbonylation was successful in the absence of any reaction solvent, and when considered along with the absence of any byproducts in the reaction, is consistent with the principles of green chemistry (Scheme 8).

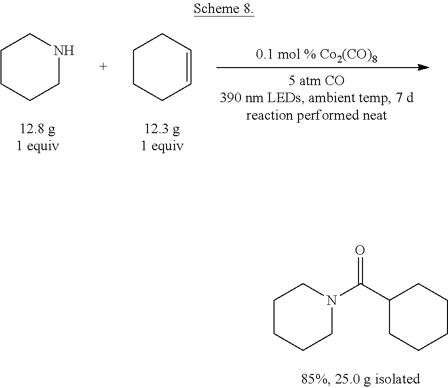 [SMILES: C1CCNCC1 ; C1=CCCCC1 ; O=C(C1CCCCC1)N1CCCCC1]

### Efficient Hydroaminocarbonylation in the Absence of Solvent

The above substrate scope of alkene and amine starting materials were even further explored to demonstrate the versatility in starting materials for this hydroaminocarbonylation (FIG. 3A and FIG. 3B).

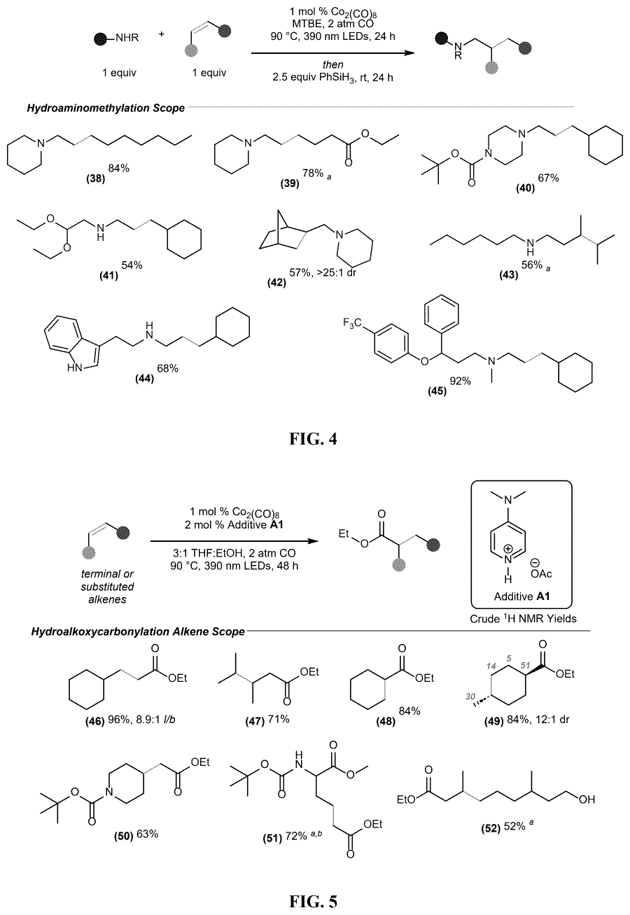

As already mentioned above, current hydroaminomethylations of alkenes for the synthesis of alkylamines, are synthesized via the sequential hydroformylation, condensation, and reduction of intermediate imines. This reaction typically requires the use of precious metal catalysts, and there are no examples using a first-row metal in reactions with alkylamines. By contrast, the methods disclosed herein comprise hydroaminocarbonylation and amide reduction to afford the same products via telescoped reduction of the amide with a silane. It is found that upon the completion of the hydroaminocarbonylation, simply adding a silane to the reaction enables a cobalt carbonyl-catalyzed amide reduction to provide alkylamine products. A representative substrate scope of the hydroaminomethylation demonstrating the successful coupling of the major classes of alkene and amines with sequential reduction to alkyl amine products is depicted in FIG. 4.

In addition, the methods disclosed herein can extend to hydroalkoxycarbonylations as well. Shown below are general reaction conditions when using an alcohol and an olefin as starting materials (Scheme 9).

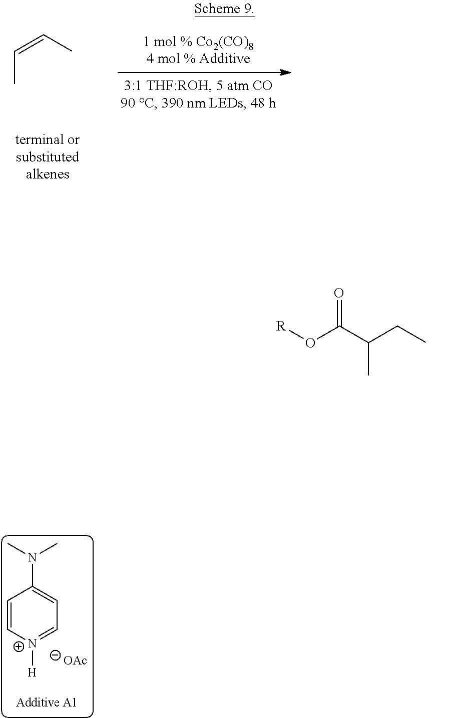

### Cobalt-Catalyzed Hydroalkoxycarbonylation Under Mild Conditions Promoted by Visible Light

A representative substrate scope of the hydroalkoxycarbonylation demonstrating the successful coupling of the major classes of alkenes is depicted in FIG. 5. With respect to alkene scope, terminal, 1,1- and 1,2-disubstituted, tri- and tetrasubstituted alkenes are all excellent substrates in the hydroalkoxycarbonylation. With internal alkene substrates, efficient chain walking can be observed, leading to selective functionalization of the end of the chain (FIG. 5, compounds 47, 49 and 52).

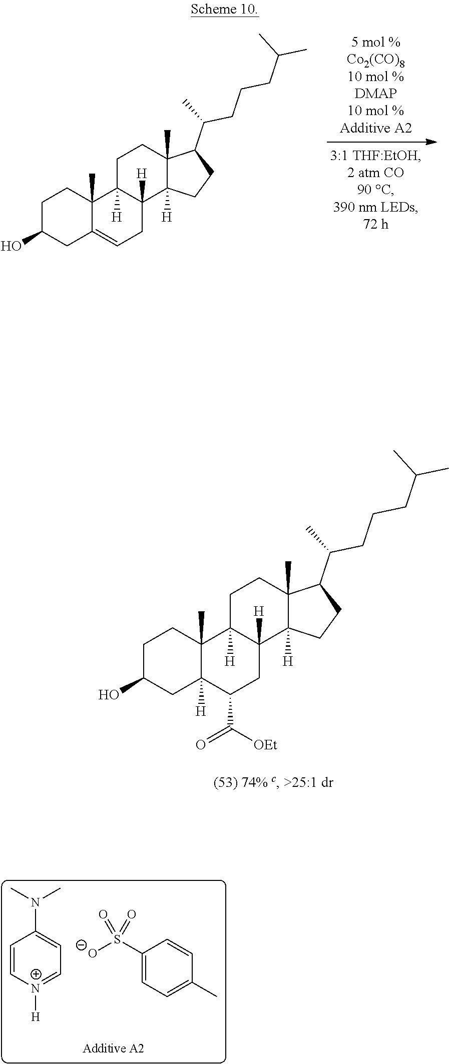

### Example of a Cobalt-Catalyzed Hydroalkoxycarbonylation of a Hindered Olefin Using Mild Conditions Promoted by Visible Light

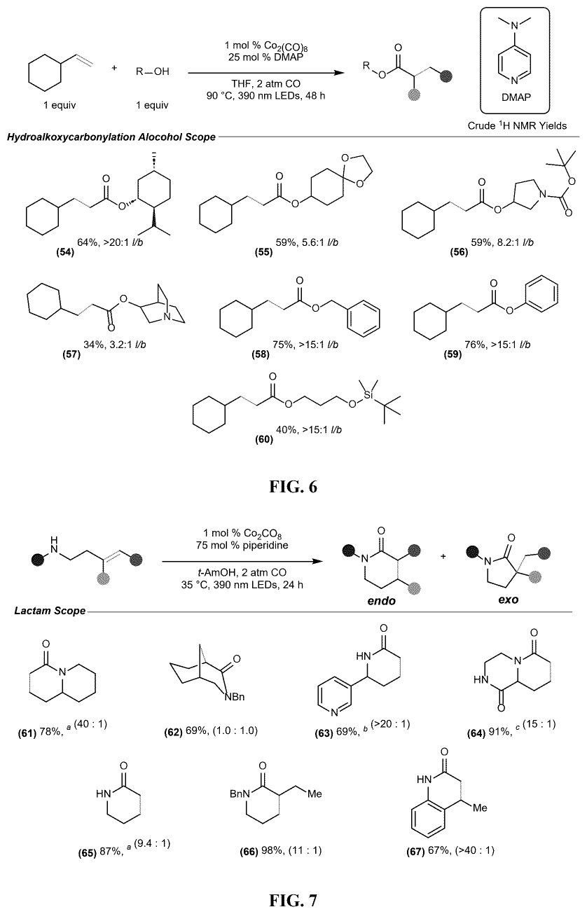

This hydroalkoxycarbonylation system is also able to produce ester products when the alcohol starting material is the limiting reagent instead of solvent. This enables the coupling of more complex alcohol that would not be practical to use in solvent quantitates. The scope with respect to the alcohol coupling partner is broad, highlighting the overall utility of the methods. Diverse primary and secondary alcohols all react efficiently in couplings with vinylcyclohexane (FIG. 6). The reaction even proceeds using water as the nucleophile to produce the formal hydrocarboxylation product (75) from vinylcyclohexane shown below in scheme 11.

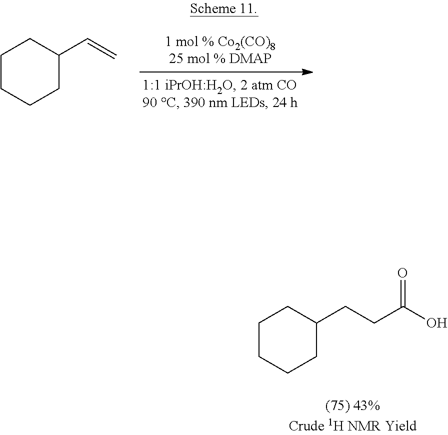

### Cobalt-Catalyzed Hydrocarboxylation of Vinylcyclohexane Using Mild Conditions Promoted by Visible Light

In addition, the methods disclosed herein comprise hydroalkoxymethylation and reductive hydroformylation to afford ether and alcohol products, respectively, via reduction of the ester products with a silane as shown in scheme 12. Representative esters product methyl 3-phenylpropanoate was reduced using conditions similar to those of the hydroalkoxycarbonylation with the addition of silane to produce ether (76) using diphenyl silane and alcohol (77) using phenyl silane.

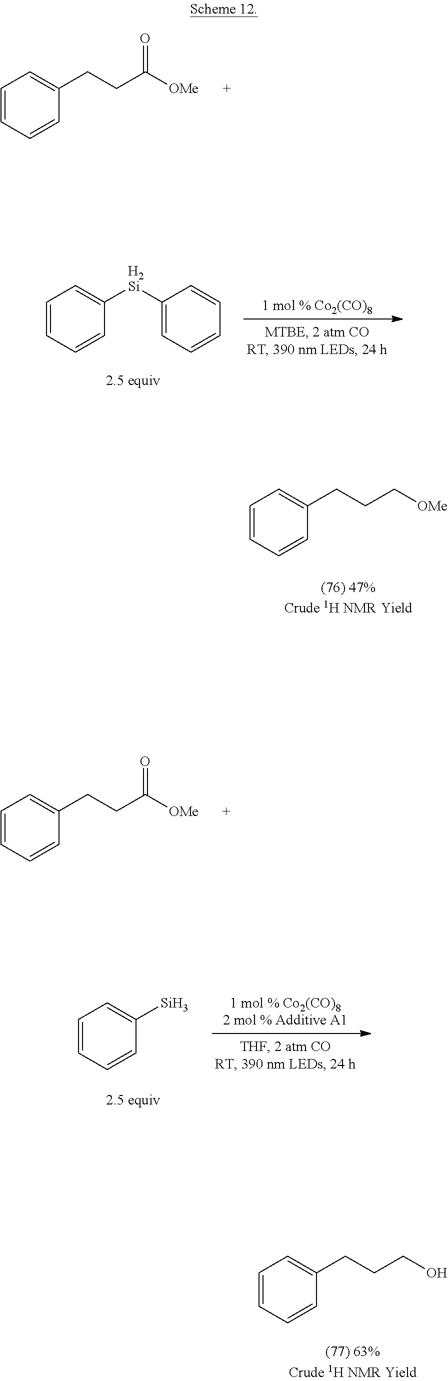

### Catalytic Reduction of Esters for Preparing Ethers and Alcohols

In addition, the methods disclosed herein can also extend to the preparation of cyclic amides and esters (lactams and lactones, respectively) as is shown in the general scheme below (Scheme 13).

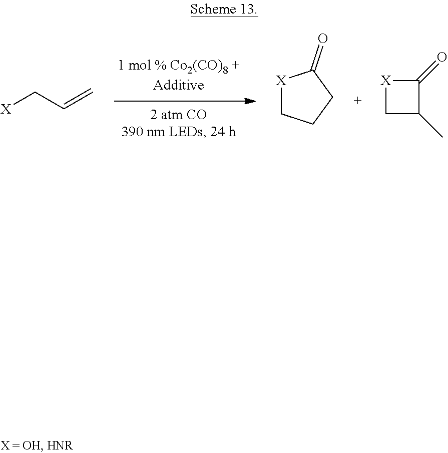

### Cobalt-Catalyzed Carbonylative Hydrofunctionalizations for Preparing Cyclic Amides and Esters; n=1-10

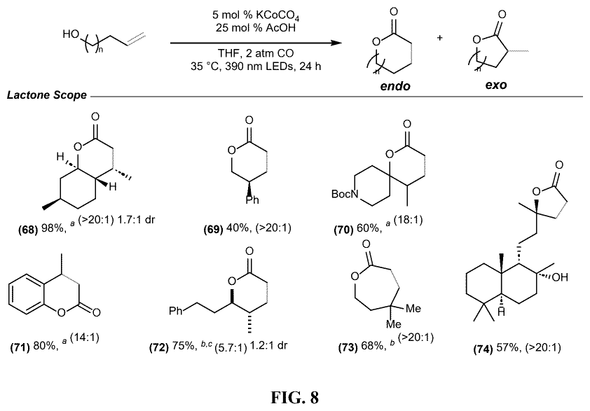

The scope with respect to the alkenes that can be used in Scheme 13 is shown in FIG. 7 and FIG. 8. In some embodiments, the olefin and amine functionality are present in the same starting material to form cyclic amides and/or polyamides. In some embodiments, the olefin and hydroxyl functionality are present in the same starting material to form cyclic esters and/or polyesters. In some embodiments, the cyclic amides and esters can be reduced according to the methods disclosed herein to afford the corresponding cyclic amine and/or ether. In some embodiments, all of these cyclic products can be macrocycles. It would be understood by a skilled artisan that the formation of cyclic products (i.e., cyclic amides and/or ester) versus non-cyclic products (i.e., polyamides and/or esters) can be modulated by the concentration of the starting material, e.g., the presence or absence of solvent. In addition, numerous additional examples are provided in FIG. 7 and FIG. 8 to further demonstrate the broad scope of the disclosed method.

In addition, the methods disclosed herein can also extend to the preparation of aldehydes as is shown in the general scheme below (Scheme 14).

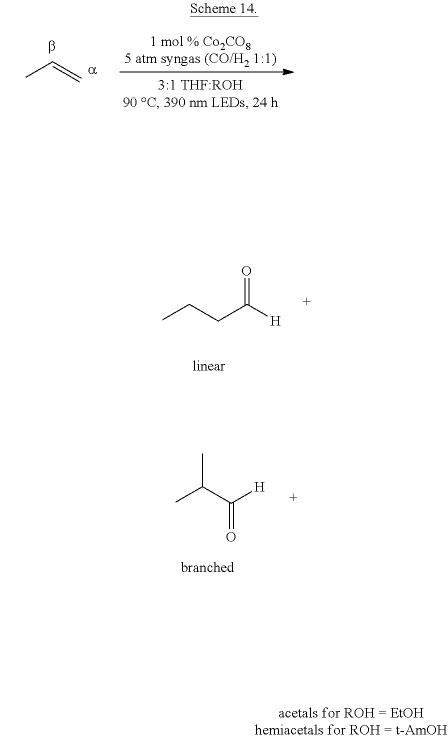

### Cobalt-Catalyzed Hydroformylation Under Mild Conditions Promoted by Visible Light

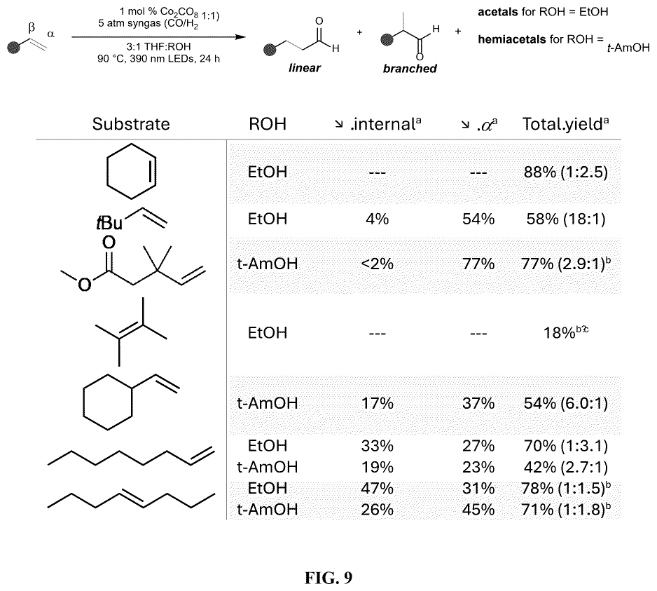

A representative substrate scope of the hydroformylation demonstrating the successful reactivity of the major classes of alkenes is depicted in FIG. 9. With respect to alkene scope, terminal, 1,1- and 1,2-disubstituted, tri- and tetrasubstituted alkenes are all good substrates in the hydroformylation.

In addition, the methods disclosed herein comprise hydroaminocarbonylation and hydroalkoxycarbonylation of alkynes to form acrylamide and acrylate products, respectively. Representative examples of alkyne reactions show below in scheme 14 yielding products (78-81) in excellent yields.

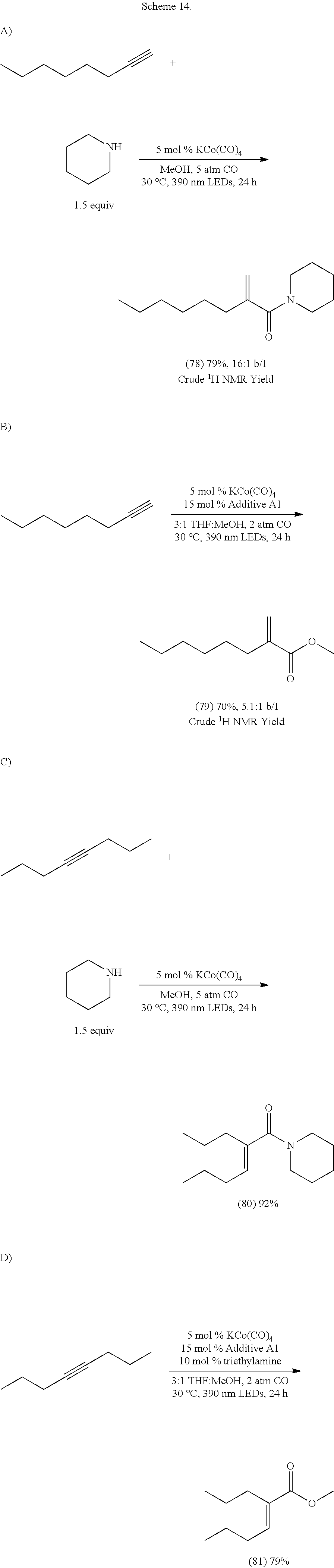

### Cobalt-Catalyzed Synthesis of Acrylamides from Alkynes with Amine Nucleophiles (A and C) and Acrylates from Alkynes with Alcohol Nucleophiles (B and D) Under Mild Conditions Promoted by Visible Light

Particular embodiments of the subject matter described herein include

1. A method of making an amide, the method comprising:
contacting an olefin with an amine in the presence of a cobalt catalyst in a carbon monoxide atmosphere while being exposed to light.

2. A method of making an acrylamide, the method comprising:
contacting an alkyne with an amine in the presence of a cobalt catalyst in a carbon monoxide atmosphere while being exposed to light.

3. The method of any one of the preceding embodiments, wherein the amine is a compound of Formula I:

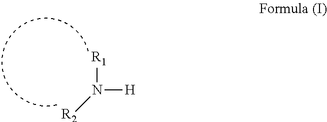

wherein R₁ and R₂ are independently selected from the group consisting of hydrogen, alkyl, cycloalkyl, heterocycloalkyl, aryl, heteroaryl, alkenyl, alkynyl, haloalkyl, heteroalkyl, bicyclic cycloalkyl, bicyclic heterocycloalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms; and wherein [symbol] represents a single bond, if present, between R₁ and R₂.

4. The method of any of the preceding embodiments, wherein R₁ and R₂ are independently selected from the group consisting of hydrogen, (C₁-C₆)alkyl, (C₃-C₆)cycloalkyl, —(CH₂)aryl, and —(CH₂)heteroaryl.

5. The method of any one of the preceding embodiments, wherein the amine is selected from the group consisting of:

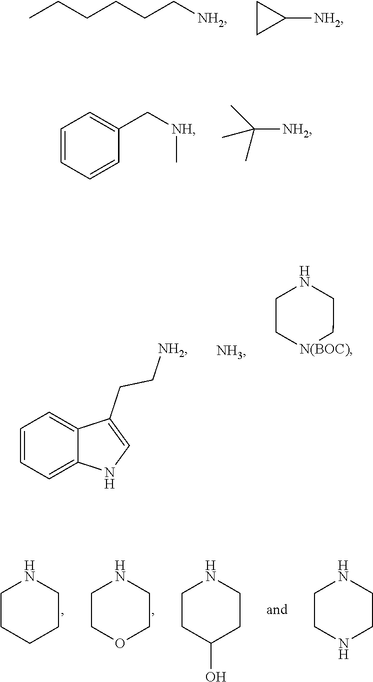

6. A method of making an ester or carboxylic acid, the method comprising:
contacting an olefin with an alcohol or water in the presence of a cobalt catalyst in a carbon monoxide atmosphere while being exposed to light.

7. A method of making an acrylate, the method comprising:
contacting an alkyne with an alcohol in the presence of a cobalt catalyst in a carbon monoxide atmosphere while being exposed to light.

8. The method of embodiments 6 or 7, wherein the alcohol is a compound of Formula Ia:

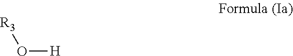

wherein R₃ is selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₘaryl, and —(CH₂)ₚheteroaryl, wherein m and p are integers independently selected from about 1 to about 20 atoms.

9. The method of embodiments 6, 7 or 8, wherein R₃ is —CH₃ (i.e., methanol).

10. A method of making an aldehyde, the method comprising:
contacting an olefin with hydrogen in the presence of a cobalt catalyst in a carbon monoxide atmosphere while being exposed to light.

11. The method of any one of embodiments 1, 3-6 and 8-10, wherein the olefin is a terminal olefin.

12. The method of any one of embodiments 1, 3-6 and 8-11, wherein the olefin is a substituted olefin selected from the group consisting of a mono-substituted olefin, a di-substituted olefin, a tri-substituted olefin, and a tetra-substituted olefin.

13. The method of any one of embodiments 1, 3-6 and 8-12, wherein the olefin is a compound of Formula II:

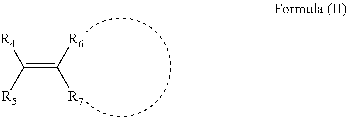

wherein R₄, R₅, R₆, and R₇ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms; and
wherein [symbol] represents a bond, if present, between R₆ and R₇.

14. The method of embodiment 13, wherein at least one of R₄, R₅, R₆, and R₇ is H.

15. The method of embodiment 14, wherein R₄ is hydrogen

16. The method of embodiment 14, wherein R₄ and R₅ are hydrogen.

17. The method of embodiment 14, wherein R₄, R₅, and R₆ are hydrogen.

18. The method of embodiment 14, wherein R₄ and R₇ are hydrogen.

19. The method of any one of embodiments 1, 3-6 and 8-12, wherein the olefin is a compound of Formula III:

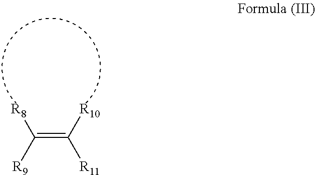

wherein R₈, R₉, R₁₀, and R₁₁ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms; and
wherein [symbol] represents a bond, if present, between R₈ and R₉.

20. The method of embodiment 19, wherein R₉ and R₁₁ are hydrogen.

21. The method of any one of embodiments 1, 3-6 and 8-20, wherein the olefin is selected from the group consisting of:

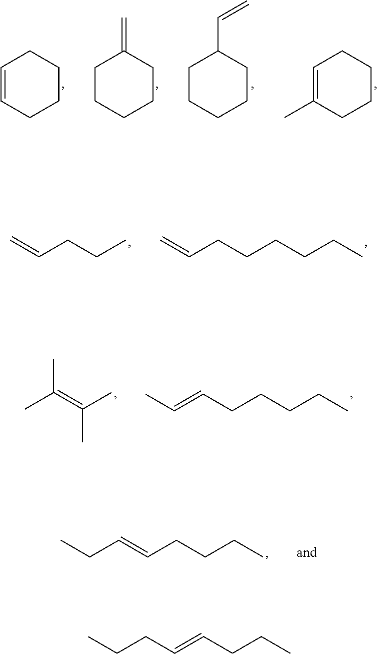

22. The method of embodiments 2 or 7, wherein the alkyne is a compound of Formula VII:

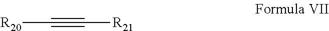

wherein R₂₀ is selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms.
wherein R₂₁ is selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms.

23. A method of making a cyclic ester, the method comprising:
mixing an olefin with a cobalt catalyst in a carbon monoxide atmosphere while being exposed to light, wherein the olefin is a functionalized olefin comprising a hydroxyl group.

24. The method of embodiment 23, wherein the olefin is a compound of Formula (II)

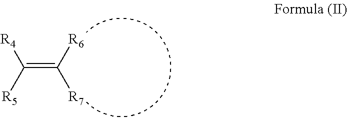

wherein R₄, R₅, R₆, and R₇ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)ₘaryl, and —(CH₂)ₚheteroaryl, wherein m, n and p are integers independently selected from about 1 to about 20 atoms;
wherein one of R₄, R₅, R₆, or R₇ is —(CH₂)ₙOH; and
wherein [symbol] represents a bond, if present, between R₆ and R₇.

25. The method of embodiment 23, wherein the olefin is a compound of Formula (III)

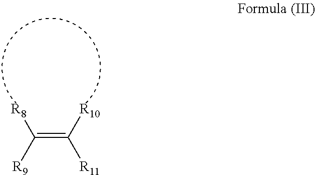

wherein R₈, R₉, R₁₀, and R₁₁ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)ₘaryl, and —(CH₂)ₚheteroaryl, wherein m, n and p are integers independently selected from about 1 to about 20 atoms;
wherein one of R₈, R₉, R₁₀, and R₁₁ is —(CH₂)ₙOH; and
wherein [symbol] represents a bond, if present between R₈ and R₁₀.

26. A method of making a cyclic amide, the method comprising:
mixing an olefin with a cobalt catalyst in a carbon monoxide atmosphere while being exposed to light, wherein the olefin is a functionalized olefin comprising an amine group.

27. The method of embodiment 26, wherein the olefin is a compound of Formula (II)

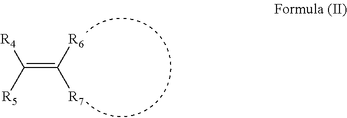

wherein R₄, R₅, R₆, and R₇ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)_{q}NH₂, —(CH₂)ₘaryl, and —(CH₂)ₚheteroaryl, wherein m, q and p are integers independently selected from about 1 to about 20 atoms;
wherein one of R₄, R₅, R₆, or R₇ is —(CH₂)_{q}NH₂; and
wherein [symbol] represents a bond, if present, between R₆ and R₇.

28. The method of embodiment 26, wherein the olefin is a compound of Formula (III)

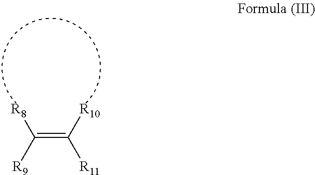

wherein R₈, R₉, R₁₀, and R₁₁ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)_{q}NH₂, —(CH₂)ₘaryl, and —(CH₂)ₚheteroaryl, wherein m, q and p are integers independently selected from about 1 to about 20 atoms;
wherein one of R₈, R₉, R₁₀, and R₁₁ is —(CH₂)_{q}NH₂; and
wherein represents a bond, if present between R₈ and R₁₀.

29. The method of any one embodiments 1, 3-6, 8-21, and 23-28, wherein the method further comprises reducing the resulting amide, ester, aldehyde, carboxylic acid, cyclic amide or cyclic ester with a reducing agent to afford the resulting amine, cyclic amine, ether, alcohol, or cyclic ether.

30. The method of any one embodiments 1, 3-6, 8-21, and 23-28, wherein the method further comprises a step of contacting the formed amide, ester, aldehyde, carboxylic acid, cyclic amide or cyclic ester with a reducing agent (e.g., diphenyl silane).

31. The method of any one of the preceding embodiments, wherein the cobalt catalyst is selected from the group consisting of Co₂(CO)₈, KCo(CO)₄, and NaCo(CO)₄.

32. The method of any one of the preceding embodiments, wherein the cobalt catalyst is present in an amount of from about 0.5 mol % to about 1.5 mol %.

33. The method of any one of embodiments 1-32, wherein the contacting step is carried out neat.

34. The method of any one of the preceding embodiments, wherein the contacting step is carried out in the presence of a solvent.

35. The method of embodiment 34, wherein the solvent is selected from the group consisting of methyl tert-butyl ether (MTBE), ethanol, isopropanol alcohol, methanol, ethyl tertiary butyl ether (ETBE), tert-amyl methyl ether (TAME), diisopropyl ether (DIPE), tetrahydrofuran (THF), toluene, tert-amyl alcohol (t-AmOH), isopropylalcohol (iPrOH), diisopropylamine, pivalonitrile, acetonitrile, ethyl acetate (EtOAc), acetone, ethanol (EtOH), methanol (MeOH), trifluoroethanol, N-methyl-2-pyrrolidone (NMP), isopropyl acetate (iPrOAc), and a combination thereof.

36. The method of embodiment 35, wherein the solvent is MTBE.

37. The method of embodiment 34, 35, or 36, wherein the solvent is present in an amount of from about 1% to about 94%.

38. The method of any one of the preceding embodiments, wherein the carbon monoxide atmosphere contains carbon monoxide from about 1 to about 2.5 atoms.

39. The method of any one of the preceding embodiments, further comprising an additive, wherein the additive is a compound of Formula I, IV, V, VI, VII as disclosed herein or a combination thereof.

### EXAMPLES

### General Methods and Materials

Proton and carbon magnetic resonance spectra (¹H NMR and ¹³C NMR) were recorded on either a Bruker AVANCE NEO 400 MHz (¹H NMR at 400 MHz and ¹³C at 101 MHz) or 600 MHz (¹H NMR at 600 MHz and ¹³C at 151 MHz) NMR spectrometer with solvent resonance as the internal standard (¹H NMR: CHCl3 at 7.260 ppm, DMSO-d6 at 2.500 ppm; ¹³C NMR: CDCl3 at 77.16 ppm, DMSO-d6 at 39.52). ¹H NMR data are reported as follows: chemical shift (ppm), multiplicity (s=singlet, br s=broad singlet, d=doublet, t=triplet, q=quartet, m=multiplet), coupling constants (Hz), and integration. High-resolution mass spectrometry samples were analyzed with a Q Exactive HF-X (ThermoFisher, Bremen, Germany) mass spectrometer with samples introduced via an electrospray source (ESI) at a flow rate of 15 μL/min in methanol. Xcalibur (ThermoFisher, Bremen, Germany) was used to analyze the data. Molecular formula assignments were determined with Molecular Formula Calculator (v 1.2.3).

Analytical thin layer chromatography (TLC) was performed on SiliaPlate 250 μm thick silica gel purchased from Silicycle. Visualization was accomplished with short-wave UV light (254 nm), or potassium permanganate stain followed by heating when necessary. Purification of the reaction products was carried out by flash chromatography using Siliaflash P60 silica gel (40-63 μm) purchased from Silicycle. Carbon monoxide, Research Purity 99.99% (part number CM R200) was purchased from Airgas. Tetrahydrofuran, diethyl ether, acetonitrile, and dichloromethane were dried by passage through a column of neutral alumina under nitrogen prior to use. t-Amyl alcohol was sparged with argon before storage over 4 Å molecular sieves in an argon filled glovebox. Co₂(CO)₁₀ was purchased from Strem Chemicals, stored in a glovebox at −30° C., and used as received. All liquid amine nucleophiles were distilled prior to being stored in a glovebox. All other reagents were obtained from commercial sources and used without further purification, unless otherwise noted. In addition, all reactions were carried out under an atmosphere of dry argon in flame or oven-dried glassware with magnetic stirring. The glass tubes used were purchased from Ace Glass and the gas quick-connect adapters were obtained from Swagelok. PR160 LED lights were purchased from Kessil and set to 100% intensity (352 mW/cm² measured from 1 cm distance). An example of the carbonylation pressurization manifold and photo-excitation setup is shown below.

CAUTION: Carbon monoxide is an odorless, colorless, tasteless, poisonous gas. The permissible exposure limit (PEL) for CO set forth by OSHA is 50 ppm for eight hours. The immediately dangerous to life or health (IDLH) value set forth by the US National Institute for Occupational Safety and Health (NIOSH) is 1200 ppm. However, if any amount of CO is detected in the laboratory, steps must be taken to stop the leakage and potential exposure to CO. All manipulations with CO must be performed in a well-ventilated and functioning fume hood. Personal CO detectors (Draeger Pac 6500 series) were used to monitor the atmosphere during these manipulations.

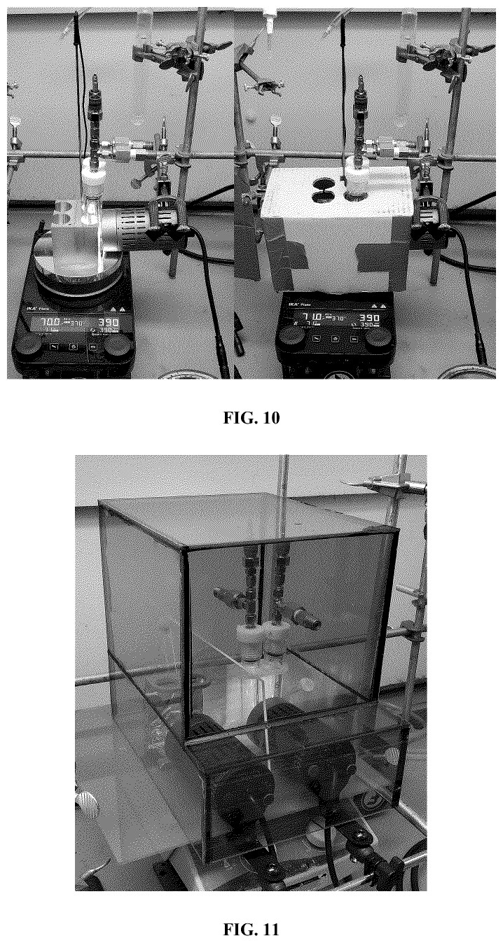

For the experimental set-up to carry out the examples below, please refer to FIGS. 10 and 11.

### Example 1: Cobalt-Catalyzed General Hydroaminocarbonylation Procedure

In a glovebox under an argon atmosphere, Co₂(CO)₈ (2.7 mg, 0.0080 mmol, 1 mol %) was combined with MTBE (2.0 mL), alkene substrate (0.80 mmol, 1.0 equiv), and amine nucleophile (0.80 mmol, 1.0 equiv) in an Ace Glass pressure tube. The vessel was sealed with a Swagelok connector cap and removed from the glovebox. Inside a fume hood with closed sashes, the tube was pressurized to 5 atm CO, purged 3 times with CO to replace argon, set to 2 atm and stirred for 24 hours at 90° C. under irradiation at 390 nm. The tube was cooled and then depressurized. Then the reaction mixture diluted with EtOAc (3 mL) and quenched with 1M HCl (3 mL). The mixture was extracted with EtOAc (3×2 mL), and the combined organic layers were allowed to sit open to air to decompose the cobalt complex as indicated by a color change from yellow to colorless (ca. 0.5-2 hours). The combined organic layers were filtered through a plug of SiO₂, eluting with EtOAc, and concentrated under reduced pressure. The crude product was purified by flash column chromatography.

### Example 2: General Hydroaminomethylation Procedure

In a glovebox under an argon atmosphere, Co₂(CO)₈ (2.7 mg, 0.0080 mmol, 1 mol %) was combined with MTBE (2.0 mL), alkene substrate (0.80 mmol, 1.0 equiv), and amine nucleophile (0.80 mmol, 1.0 equiv) in an Ace Glass pressure tube. The vessel was sealed with a Swagelok connector cap and removed from the glovebox. Inside a fume hood with closed sashes, the tube was pressurized to 5 atm CO, purged 3 times with CO to replace argon, set to 2 atm and stirred for 24 hours at 90° C. under irradiation at 390 nm. The tube was allowed to cool and then depressurized. It was then quickly opened and phenylsilane (0.25 mL, 2.0 mmol, 2.5 equiv) was added before resealing the tube, pressurizing the reaction to 2 atm of CO as previously indicated, and stirring for another 24 hours under irradiation at 390 nm. The tube was depressurized and the mixture was allowed to stir with SiO₂ (1 g) and EtOAc (3 mL) for 1 hour. This mixture was then filtered through a plug of SiO₂ eluting with 1% triethylamine/15% MeOH/84% DCM. After concentrating under reduced pressure, the crude product was purified by flash column chromatography.

### Example 3: Cobalt-Catalyzed General Hydroalkoxycarbonylation Procedure

In a glovebox under an argon atmosphere, Co₂(CO)₈ (2.7 mg, 0.0080 mmol, 1 mol %) and additive A1 (5.8 mg, 0.032 mmol, 4 mol %) was combined with THF (1.5 mL), EtOH (0.5 mL), and alkene substrate (0.80 mmol, 1.0 equiv) in an Ace Glass pressure tube. The vessel was sealed with a Swagelok connector cap and removed from the glovebox. Inside a fume hood with closed sashes, the tube was pressurized to 5 atm CO, purged 3 times with CO to replace argon, set to 2 atm and stirred for 24 hours at 90° C. under irradiation at 390 nm. The tube was cooled and then depressurized. Then the reaction mixture diluted with Et₂O (3 mL) and allowed to sit open to air to decompose the cobalt complex as indicated by a color change from purple to brown (ca. 0.5-2 hours). The combined organic layers were filtered through a plug of SiO₂, eluting with Et₂O, and concentrated under reduced pressure. The crude product was purified by flash column chromatography.

### Example 4: Cobalt-Catalyzed General Hydroformylation Procedure

In a glovebox under an argon atmosphere, Co₂(CO)₈ (2.7 mg, 0.0080 mmol, 1 mol %) was combined with THF (1.5 mL), ROH (0.5 mL), and alkene substrate (0.80 mmol, 1.0 equiv) in an Ace Glass pressure tube. The vessel was sealed with a Swagelok connector cap and removed from the glovebox. Inside a fume hood with closed sashes, the tube was pressurized to 5 atm CO, purged 3 times with CO to replace argon, set to 5 atm and stirred for 5 minutes at 90° C. under irradiation at 390 nm. Then, the tube was pressurized to 5 atm of syngas (1:1 CO:H₂), purged 3 times with syngas to replace the CO, set to 5 atm and stirred for 24 hours at 90° C. under irradiation at 390 nm. The tube was cooled and then depressurized. Then the reaction mixture diluted with Et₂O (3 mL) and allowed to sit open to air to decompose the cobalt complex as indicated by a color change from purple to brown (ca. 0.5-2 hours). The combined organic layers were filtered through a plug of SiO₂, eluting with Et₂O, and concentrated under reduced pressure. Hexamethyldisiloxane (20 μL) is added to the crude reaction mixture for analysis by ¹H NMR.

What is claimed is:

1. A method of making an amide, the method comprising:
contacting an olefin with an amine in the presence of a cobalt catalyst in a carbon monoxide atmosphere while being exposed to light.

2. The method of claim 1, wherein the amine is a compound of Formula I: 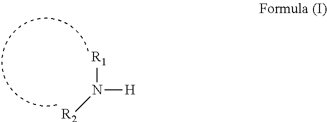
wherein R₁ and R₂ are independently selected from the group consisting of hydrogen, alkyl, cycloalkyl, heterocycloalkyl, aryl, heteroaryl, alkenyl, alkynyl, haloalkyl, heteroalkyl, bicyclic cycloalkyl, bicyclic heterocycloalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms; and wherein [symbol] represents a single bond, if present, between R₁ and R₂.

3. The method of claim 2, wherein R₁ and R₂ are independently selected from the group consisting of hydrogen, (C₁-C₆)alkyl, (C₃-C₆)cycloalkyl, —(CH₂)aryl, and —(CH₂)heteroaryl.

4. The method of claim 2, wherein the amine is selected from the group consisting of:

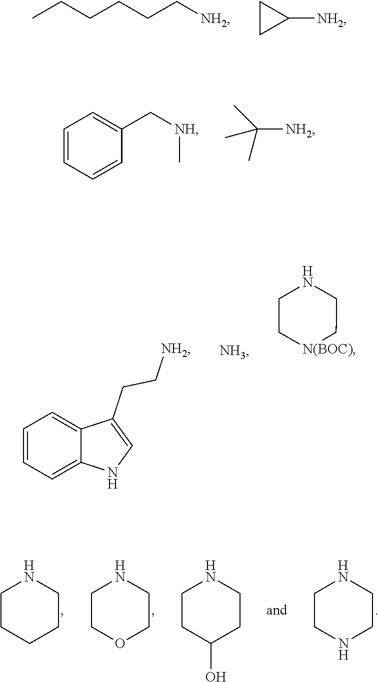

5. The method of claim 1, wherein the olefin is a terminal olefin or is a substituted olefin selected from the group consisting of a mono-substituted olefin, a di-substituted olefin, a tri-substituted olefin, and a tetra-substituted olefin.

6. (canceled)

7. The method of claim 1, wherein the olefin is a compound of Formula II: 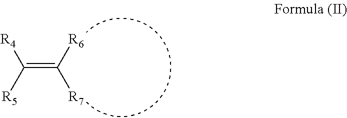
wherein R₄, R₅, R₆, and R₇ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms; and
wherein [symbol] represents a bond, if present, between R₆ and R₇.

8. The method of claim 7, wherein at least one of R₄, R₅, R₆, and R₇ is H.

9. The method of claim 7, wherein R₄ is hydrogen

10. The method of claim 7, wherein R₄ and R₅ are hydrogen or R₄ and R₇ are hydrogen.

11. The method of claim 7, wherein R₄, R₅, and R₆ are hydrogen.

12. (canceled)

13. The method of claim 1 wherein the olefin is a compound of Formula III: 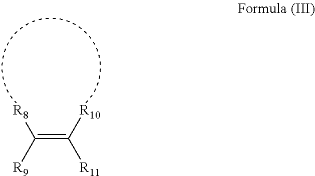
wherein R₈, R₉, R₁₀, and R₁₁ are independently selected from the group consisting of hydrogen, alkyl, cycloalkyl, heterocycloalkyl, aryl, heteroaryl, alkenyl, alkynyl, haloalkyl, heteroalkyl, cycloalkenyl, —(CH₂)ₙOH, —(CH₂)_{q}NH₂, —(CH₂)ₘaryl, and —(CH₂)ₚheteroaryl, wherein m, n, q and p are integers independently selected from about 1 to about 20 atoms; and
wherein [symbol] represents a bond, if present, between R₈ and R₉.

14. The method of claim 13, wherein R₉ and R₁₁ are hydrogen.

15. The method of claim 13, wherein the olefin is selected from the group consisting of:

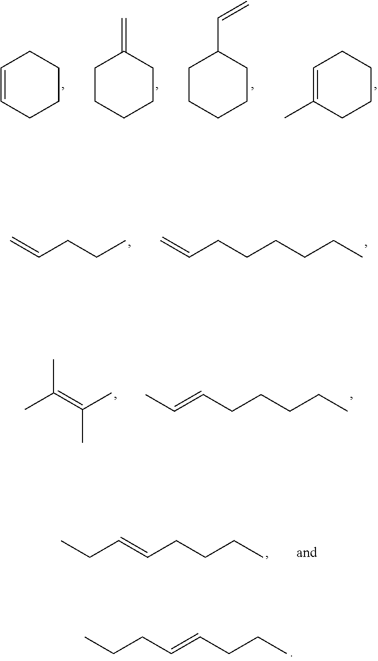

16. The method of claim 1, wherein the cobalt catalyst is selected from the group consisting of Co₂(CO)₈, KCo(CO)₄, and NaCo(CO)₄.

17. The method of claim 1, wherein the cobalt catalyst is present in an amount of from about 0.5 mol % to about 1.5 mol %.

18. The method of claim 1, wherein the contacting step is carried out neat.

19. The method of claim 1, wherein the contacting step is carried out in the presence of a solvent selected from the group consisting of methyl tert-butyl ether (MTBE), ethanol, isopropanol alcohol, methanol, ethyl tertiary butyl ether (ETBE), tert-amyl methyl ether (TAME), diisopropyl ether (DIPE), tetrahydrofuran (THF), toluene, tert-amyl alcohol (t-AmOH), isopropylalcohol (iPrOH), diisopropylamine, pivalonitrile, acetonitrile, ethyl acetate (EtOAc), acetone, ethanol (EtOH), methanol (MeOH), trifluoroethanol, N-methyl-2-pyrrolidone (NMP), isopropyl acetate (iPrOAc), and a combination thereof.

20. (canceled)

21. The method of claim 20, wherein the solvent is MTBE.

22. The method of claim 19, wherein the solvent is present in an amount of from about 1% to about 94%.

23. The method of claim 1, wherein the carbon monoxide atmosphere contains carbon monoxide from about 1 to about 2.5 atoms.
